(19) United States
(12) Patent Application Publication
(10) Pub. No.: US 2008/0176866 A1
(43) Pub. Date: Jul. 24, 2008

(54) NOVEL HETARYL-PHENYLENEDIAMINE-PYRIMIDINES AS PROTEIN KINASE INHIBITORS

(76) Inventors: Rolf Jautelat, Glienicke/Nordbahn (DE); Gerhard Siemeister, Berlin (DE); Ulrich Luecking, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/960,118

(22) Filed: Dec. 19, 2007

Related U.S. Application Data
(60) Provisional application No. 60/880,031, filed on Jan. 12, 2007.

(30) Foreign Application Priority Data
Dec. 20, 2006 (EP) 06077236.5

(51) Int. Cl. A61K 31/495; C07D 239/28; A61P 35/00

(52) U.S. Cl. 514/256; 544/319; 544/328; 514/275

(57) ABSTRACT

The invention relates to novel hetaryl-phenylenediamine-pyrimidines and to their structurally related oxygen and sulphur analogues of the general formula I, processes for their preparation, and their use as medicaments.

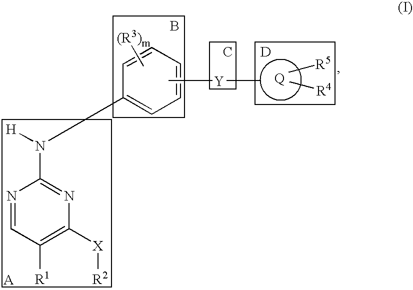

This application claims the benefit of the filing date of U.S. Provisional Application Ser. No. 60/880,031 filed Jan. 12, 2007.

The invention relates to novel hetaryl-phenylenediamine-pyrimidines and to their structurally related oxygen and sulphur analogues as protein kinase inhibitors.

Many biological processes such as, for example, DNA replication, energy metabolism, cell growth or cell differentiation in eukaryotic cells are regulated by reversible phosphorylation of proteins. The degree of phosphorylation of a protein has an influence inter alia on the function, localization or stability of proteins. The enzyme families of protein kinases and protein phosphatases are responsible respectively for the phosphorylation and dephosphorylation of proteins.

It is hoped, through inhibition of specific protein kinases or protein phosphatases, to be able to intervene in biological processes in such a way that causal or symptomatic treatment of diseases of the human or animal body is possible.

Protein kinases are of particular interest in this connection, inhibition thereof making the treatment of cancer possible.

The following protein kinase families come under consideration for example as targets for inhibitory molecules:
a) Cell cycle kinases, i.e. kinases whose activity control the progression of the cycle of cell division. Cell cycle kinases include substantially the cyclin-dependent kinases (cdk), the polo-like kinases (Plk), and the Aurora kinases.
b) Receptor tyrosine kinases which regulate angiogenesis (angiogenic receptor tyrosine kinases), such as, for example, the receptor tyrosine kinases which are involved in the vascular endothelial growth factor (VEGF)/VEGF receptor system, fibroblast growth factor (FGF)/FGF receptor system, in the Eph ligand/EphB4 system, and in the Tie ligand/Tie system,
c) receptor tyrosine kinases whose activity contributes to the proliferation of cells (proliferative receptor tyrosine kinases), such as, for example, receptor tyrosine kinases which are involved in the platelet-derived growth factor (PDGF) ligand/PDGF receptor system, epithelial growth factor (EGF) ligand/EGF receptor system, c-Kit ligand/c-Kit receptor system and in the FMS-like tyrosine kinase 3 (Flt-3) ligand/Flt-3 system,
d) checkpoint kinases which monitor the ordered progression of cell division, such as, for example, ATM and ATR, Chk1 and Chk2, Mps1, Bub1 and BubR1,
e) kinases whose activity protects the cell from apoptosis (anti-apoptotic kinases, kinases in so-called survival pathways, anti-apoptotic kinases), such as, for example, Akt/PKB, PDK1, IkappaB kinase (IKK), Pim1, and integrin-linked kinase (ILK),
f) kinases which are necessary for the migration of tumour cells (migratory kinases), such as, for example, focal adhesion kinase (FAK) and Rho kinase (ROCK).

Inhibition of one or more of these protein kinases opens up the possibility of inhibiting tumour growth.

In this connection there is a need in particular for structures which, besides inhibiting cell cycle kinases, inhibit tumour growth through the inhibition of one or more further kinases (multi-target tumour growth inhibitors=MTGI). It is particularly preferred to inhibit in addition receptor tyrosine kinases which regulate angiogenesis.

The structures of the following patent applications form the structurally close prior art:

WO 2002/096888 discloses anilinopyrimidine derivatives as inhibitors of cyclin-dependent kinases. Hetaryl-phenylenediamine-pyrimidines are not disclosed.

WO 2004/026881 discloses macrocyclic anilinopyrimidine derivatives as inhibitors of cyclin-dependent kinases. Hetaryl-phenylenediamine-pyrimidines are not disclosed.

WO 2005/037800 discloses open anilinopyrimidine derivatives as inhibitors of cyclin-dependent kinases. Hetaryl-phenylenediamine-pyrimidines are not disclosed.

WO 2004/0046118 discloses open diphenylaminopyrimidines for the treatment of diseases associated with hyperproliferation. Hetaryl-phenylenediamine-pyrimidines are not disclosed.

It is common to all these structures of the prior art that they inhibit cell cycle kinases.

Starting from this prior art, it is the object of the present invention to provide alternative protein kinase inhibitors.

In particular, the object of the present invention is to provide inhibitors of protein kinases by which tumour growth can be inhibited.

There is a need in particular for compounds which, besides cell cycle kinases, also inhibit receptor tyrosine kinases which inhibit angiogenesis.

There is a need in particular for compounds which, besides inhibiting one or more protein kinases, in fact inhibits the proliferation of cancer cells.

The object of the present application is achieved by compounds of the general formula (I) with building blocks A, B, C and D,

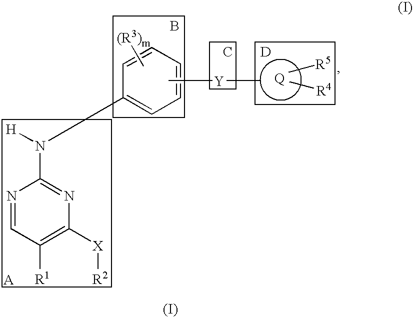

in which
R¹ is halogen, —CF₃, —OCF₃, C₁-C₄-alkyl or nitro,
R² is a C₁-C₁₀-alkyl, C₂-C₁₀-alkenyl or C₂-C₁₀-alkynyl radical, a C₃-C₇-cycloalkyl or a heterocyclyl ring having 3 to 7 ring atoms, in each case optionally substituted one or more times, identically or differently, by hydroxy, —NR⁸R⁹, —NR⁷—C(O)—R¹², —NR⁷—C(O)—OR¹², —NR⁷—C(O)—NR⁸R⁹ or a monocyclic or bicyclic heteroaryl which is optionally itself substituted one or more times by hydroxy or a C₁-C₆-alkyl radical.
R³ is
   (i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹ or —NR⁷—SO₂—R¹² and/or
   (ii) a C₁-C₅-alkyl and/or C₁-C₅-alkoxy radical which is optionally substituted one or more times, identically or differently, by halogen, hydroxy, C₁-C₆-alkoxy, —CF₃, —OCF₃ or —NR⁸R⁹,
m is 0-3,
R⁴ and R⁵ are independently of one another
   (i) hydrogen, —NHR⁸, —OR⁸, halogen, —(CO)—NR⁸R⁹ and/or
   (ii) a C₁-C₄-alkyl, C₃-C₅-alkenyl, C₃-C₅-alkynyl radical or a C₃-C₆-cycloalkyl ring, in each case optionally themselves substituted one or more times, identically or differently, by hydroxy, —NR³R⁹, cyano, halogen, —CF₃, C₁-C₆-alkoxy, —OCF₃ and/or C₁-C₆-alkyl
X and Y are independently of one another
   —O—, —S—, —S(O)—, —S(O)₂— or —NR¹⁵—, where
   R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃, or
   if X is —NR¹⁵—,
   —NR¹⁵— and R² preferably alternatively together form a 3 to 6 membered ring which, in addition to the nitrogen atom, optionally comprises a further heteroatom, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 or 2 double bonds, and/or is interrupted by a —C(O) group.
Q is a monocyclic or bicyclic heteroaryl ring,
R⁶ is
   (i) hydrogen or
   (ii) a C₁-C₄-alkyl, C₃-C₅-alkenyl, C₃-C₅-alkynyl or C₁-C₅-alkoxy radical, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.
R⁷ is hydrogen,
R⁸ and R⁹ are independently of one another
   hydrogen and/or a C₁-C₅-alkyl, C₂-C₅-alkenyl radical, a C₃-C₇-cycloalkyl and/or phenyl ring and/or a monocyclic heteroaryl ring, in each case optionally substituted one or more times, identically or differently, by hydroxy, —NR¹⁰R¹¹, —NR⁷—C(O)—R¹² and/or C₁-C₆-alkoxy,
   or
R⁸ and R⁹ form together with the nitrogen atom a 5- to 7-membered ring which, in addition to the nitrogen atom, optionally contains 1 further heteroatom and which may be substituted one or more times, identically or differently, by hydroxy, —NR¹⁰R¹¹ and/or C₁-C₆-alkoxy,
R¹⁰ and R¹¹ are independently of one another hydrogen or a C₁-C₆-alkyl radical which is optionally substituted one or more times, identically or differently, by hydroxy, halogen or C₁-C₆-alkoxy,
R¹² is a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,
   in each case optionally themselves substituted one or more times, identically or differently, by hydroxy, halogen, nitro, —NR⁸R⁹, C₁-C₆-alkyl and/or C₁-C₆-alkoxy.
R¹³ and R¹⁴ are independently of one another a C₁-C₆-alkyl, C₂-C₆-alkenyl and/or C₂-C₆-alkynyl radical, a C₃-C₇-cycloalkyl and/or phenyl ring, a heterocyclyl ring having 3 to 8 ring atoms and/or a monocyclic heteroaryl ring, in each case optionally themselves substituted one or more times, identically or differently, by hydroxy, —NR⁸R⁹ and/or C₁-C₆-alkoxy.
R¹⁶ is a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
   and the salts, diastereomers and enantiomers thereof.

No prior art document discloses the compounds according to the invention or makes them obvious.

The following definitions underlie the invention:

### Cₙ-Alkyl:

Monovalent, straight-chain or branched, saturated hydrocarbon radical having n carbon atoms.

A C₁-C₆ alkyl radical includes inter alia for example:

methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-1,2-dimethylbutyl-.

A methyl, ethyl, propyl or isopropyl radical is preferred.

### Cₙ-Alkenyl:

Monovalent, straight-chain or branched hydrocarbon radical having n carbon atoms and at least one double bond.

A C₂-C₁₀ alkenyl radical includes inter alia for example:

vinyl-, allyl-, (E)-2-methylvinyl-, (Z)-2-methylvinyl-, homoallyl-, (E)-but-2-enyl-, (Z)-but-2-enyl-, (E)-but-1-enyl-, (Z)-but-1-enyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, isopropenyl-, 2-methylprop-2-enyl-, 1-methylprop-2-enyl-, 2-methylprop-1-enyl-, (E)-1-methylprop-1-enyl-, (Z)-1-methylprop-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, (E)-3,3-dimethylprop-1-enyl-, (Z)-3,3-dimethylprop-1-enyl-, 1-(1,1-dimethylethyl)ethenyl.

A vinyl or allyl radical is preferred.

### Cₙ-Alkynyl:

Monovalent, straight-chain or branched hydrocarbon radical having n carbon atoms and at least one triple bond.

A C₂-C₁₀ alkynyl radical includes inter alia for example:

ethynyl-, prop-1-ynyl-, prop-2-ynyl-, but-1-ynyl-, but-2-ynyl-, but-3-ynyl-, pent-1-ynyl-, pent-2-ynyl-, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- or a 3,3-dimethylbut-1-ynyl-.

An ethynyl, prop-1-ynyl or prop-2-ynyl radical is preferred.

### Cₙ-Cycloalkyl:

Monovalent, cyclic hydrocarbon ring having n carbon atoms.

C₃-C₇-Cycloalkyl ring includes:

cyclopropyl, cyclobutyl, cyclopentyl, cyclohexyl and cycloheptyl.

A cyclopropyl, cyclopentyl or a cyclohexyl ring is preferred.

### Cₙ-Alkoxy:

Straight-chain or branched Cₙ-alkyl ether residue of the formula —OR with R=alkyl.

### Cₙ-Aryl

Cₙ-Aryl is a monovalent, aromatic ring system without heteroatom having n hydrocarbon atoms.

C₆-Aryl is identical to phenyl. C₁₀-Aryl is identical to naphthyl.

Phenyl is preferred.

### Heteroatoms

Heteroatoms are to be understood to include oxygen, nitrogen or sulphur atoms.

### Heteroaryl

Heteroaryl is a monovalent, aromatic ring system having at least one heteroatom different from a carbon. Heteroatoms which may occur are nitrogen atoms, oxygen atoms and/or sulphur atoms. The valence bond may be on any aromatic carbon atom or on a nitrogen atom. A nitrogen atom as heteroatom may be present in oxidized form as N-oxide.

A monocyclic heteroaryl ring according to the present invention has 5 or 6 ring atoms.

Heteroaryl rings having 5 ring atoms include for example the rings:

thienyl, thiazolyl, furanyl, pyrrolyl, oxazolyl, imidazolyl, pyrazolyl, isoxazolyl, isothiazolyl, oxadiazolyl, triazolyl, tetrazolyl and thiadiazolyl.

Heteroaryl rings having 6 ring atoms include for example the rings:

pyridyl, pyridazinyl, pyrimidinyl, pyrazinyl and triazinyl.

A bicyclic heteroaryl ring according to the present invention has 9 or 10 ring atoms.

Heteroaryl rings having 9 ring atoms include for example the rings:

phthalidyl-, thiophthalidyl-, indolyl-, isoindolyl-, indazolyl-, benzothiazolyl-, indolonyl-, isoindolonyl-, benzofuranyl, benzothienyl, benzimidazolyl, benzoxazolyl, azocinyl, indolizinyl, purinyl.

Heteroaryl rings having 10 ring atoms include for example the rings:

isoquinolinyl-, quinolinyl-, benzoxazinonyl-, phthalazinonyl, quinolonyl-, isoquinolonyl-, quinazolinyl-, quinoxalinyl-, cinnolinyl-, phthalazinyl-, 1,7- or 1,8-naphthyridinyl-, quinolinyl-, isoquinolinyl-, quinazolinyl- or quinoxalinyl-

Monocyclic heteroaryl rings having 5 or 6 ring atoms are preferred.

### Heterocyclyl

Heterocyclyl in the context of the invention is a completely hydrogenated heteroaryl (completely hydrogenated heteroaryl=saturated heterocyclyl), i.e. a non-aromatic ring system having at least one heteroatom different from a carbon. Heteroatoms which may occur are nitrogen atoms, oxygen atoms and/or sulphur atoms. The valence bond may be on any carbon atom or on a nitrogen atom.

Heterocyclyl ring having 3 ring atoms includes for example:

aziridinyl.

Heterocyclyl ring having 4 ring atoms includes for example:

azetidinyl, oxetanyl.

Heterocyclyl rings having 5 ring atoms include for example the rings:

pyrrolidinyl, imidazolidinyl, pyrazolidinyl and tetrahydrofuranyl.

Heterocyclyl rings having 6 ring atoms include for example the rings:

piperidinyl, piperazinyl, morpholinyl, tetrahydropyranyl and thiomorpholinyl.

Heterocyclyl ring having 7 ring atoms includes for example:

azepanyl, oxepanyl, [1,3]-diazepanyl, [1,4]-diazepanyl.

Heterocyclyl ring having 8 ring atoms includes for example:

oxocanyl, azocanyl.

### Halogen

The term halogen includes fluorine, chlorine, bromine and iodine. Bromine is preferred.

### Building Block A

R¹ in the general formula (I) may be:

halogen, —CF₃, —OCF₃, C₁-C₄-alkyl or nitro.

R¹ is preferably halogen, —CF₃ or C₁-C₂-alkyl.

R¹ is more preferably halogen or —CF₃. R¹ is particularly preferably halogen, especially bromine.

R² in the general formula (I) may be:

a C₁-C₁₀-alkyl, C₂-C₁₀-alkenyl or C₂-C₁₀-alkynyl radical, a C₃-C₇-cycloalkyl or a heterocyclyl ring having 3 to 7 ring atoms, in each case optionally substituted one or more times, identically or differently, by hydroxy, —NR⁸R⁹, —NR⁷—C(O)—R¹²,

—NR⁷—C(O)—OR¹², —NR⁷—C(O)—NR⁸R⁹ or a monocyclic or bicyclic heteroaryl which is optionally itself substituted one or more times by hydroxy or a C₁-C₆-alkyl radical.

R² is preferably:

a C₁-C₈-alkyl, C₂-C₈-alkenyl or C₂-C₈-alkynyl radical, a C₃-C₆-cycloalkyl or a heterocyclyl ring having 3 to 5 ring atoms, in each case optionally substituted one or more times, identically or differently, by hydroxy, —NR⁸R⁹, —NR⁷—C(O)—R¹², —NR⁷—C(O)—OR¹², —NR⁷—C(O)—NR⁸R⁹ or a monocyclic heteroaryl which is optionally itself substituted one or more times by hydroxy or a C₁-C₅-alkyl radical.

R² is more preferably:

a C₁-C₆-alkyl, identically or differently substituted by hydroxy, —NR⁸R⁹, —NR⁷—C(O)—R¹² or a monocyclic heteroaryl which is optionally itself substituted one or more times by a C₁-C₅-alkyl radical.

R² is particularly preferably:

a C₁-C₆-alkyl identically or differently substituted by hydroxy.

X in the general formula (I) may be:

—O—, —S—, —S(O)—, —S(O)₂— or —NR¹⁵—, where

R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃,

or
if X is —NR¹⁵—,
—NR¹⁵— and R² preferably alternatively together form a 3 to 6 membered ring which, in addition to the nitrogen atom, optionally comprises a further heteroatom, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 or 2 double bonds, and/or is interrupted by a —C(O) group.
X is preferably —O— or —NR¹⁵—,
   where,
R¹⁵ is hydrogen or a C₃-C₆-alkyl radical, C₃-C₇-cycloalkyl or a heterocyclyl ring having 3 to 6 ring atoms, in each case optionally substituted one or more times, identically or differently, by hydroxy, —NR¹⁰R¹¹, cyano, halogen, —CF₃, C₁-C₆-alkoxy and/or —OCF₃,
   or
if X is —NR¹⁵—,
—NR¹⁵— and R² more preferably alternatively together form a 5 or 6 membered ring which, in addition to the nitrogen atom, optionally comprises a further heteroatom and is optionally substituted one or more times, identically or differently, by hydroxy, C₁-C₆-alkyl, C₁-C₆-alkoxy, —C(O)R¹², —SO₂R¹², halogen or the group —NR⁸R⁹.
X is particularly preferably —O— or —NR¹⁵—, where R¹⁵ is hydrogen.
X is very particularly preferably —O—

### Building Block B

R³ in the general formula (I) may be:
(i) hydroxy, halogen, cyano, nitro, —CF₃, —OCF₃, —C(O)NR⁸R⁹, —C(S)NR⁸R⁹, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₅-alkyl and/or C₁-C₅-alkoxy radical which is optionally substituted one or more times, identically or differently, by halogen, hydroxy, C₁-C₆-alkoxy, —CF₃, —OCF₃ or —NR⁸R⁹,
   R³ is preferably
(i) hydroxy, halogen, cyano, nitro, —CF₃, —OCF₃, —NR⁸R⁹, —NR⁷—C(O)—R¹², —NR⁷—C(O)—OR¹², —NR⁷—C(O)—NR⁸R⁹, —NR⁷—SO₂—R¹², and/or
(ii) a C₁-C₃-alkyl and/or C₁-C₃-alkoxy radical which is optionally substituted one or more times, identically or differently, by halogen, hydroxy, C₁-C₆-alkoxy, —CF₃, —OCF₃ or —NR⁸R⁹

R³ is more preferably:
(i) hydroxy, halogen, cyano, nitro, —CF₃, —OCF₃, —NR⁸R⁹ and/or
(ii) a C₁-C₃-alkyl and/or C₁-C₃-alkoxy radical

R³ is particularly preferably:

halogen, is a C₁-C₃-alkyl and/or C₁-C₃-alkoxy radical and here in particular is fluorine, chlorine, methyl and/or methoxy.

In the general formula (I), m may be:

0-3, preferably 0-2, more preferably 0 or 1.

### Building Block C

Y in the general formula (I) may be:

—O—, —S—, —S(O)—, —S(O)₂— or —NR¹⁵—, where

R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃, or

if X is —NR¹⁵—,

—NR¹⁵— and R² preferably alternatively together form a 3 to 6 membered ring which, in addition to the nitrogen atom, optionally comprises a further heteroatom, is optionally substituted one or more times, identically or differently, by hydroxy, C₁-C₆-alkyl, C₁-C₆-alkoxy, —C(O)R¹², —SO₂R¹², halogen or the group —NR⁸R⁹, optionally comprises 1 or 2 double bonds, and/or is interrupted by a —C(O) group.

Y is preferably:

—O—, —S— or —NR¹⁵—, where

R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃.

Y is more preferably —O— or —NR¹⁵—,

where

R¹⁵ is hydrogen or a C₁-C₃-alkyl radical, C₃-C₇-cycloalkyl or a heterocyclyl ring having 3 to 6 ring atoms, in each case optionally substituted one or more times, identically or differently, by hydroxy, —NR¹⁰R¹¹, cyano, halogen, —CF₃, C₁-C₆-alkoxy and/or —OCF₃.

Y is particularly preferably —O— or —NR¹⁵—, where R¹⁵ is hydrogen or a C₁-C₃-alkyl radical.

Y is very particularly preferably —NR¹⁵—, where R¹⁵ is hydrogen or a C₁-C₃-alkyl radical.

### Building Block D

Q in the general formula (I) may be:

a monocyclic or bicyclic heteroaryl ring.

Q is preferably a monocyclic heteroaryl ring.

Q is more preferably a monocyclic heteroaryl ring having 6 ring atoms.

If Q is a monocyclic heteroaryl ring having 6 ring atoms, a pyrimidinyl, pyridyl or pyridyl N-oxide ring is preferred.

If Q is a monocyclic heteroaryl ring having 5 ring atoms, a tetrazolyl or triazolyl ring is preferred.

If Q is a bicyclic heteroaryl ring, an indolyl or benzothiazolyl ring is preferred.

R⁴ and R⁵ in the general formula (I) may be independently of one another:
(i) hydrogen, —NHR⁸, —OR⁸, halogen, —(CO)—NR⁸R⁹ and/or
(ii) a C₁-C₄-alkyl, C₃-C₅-alkenyl, C₃-C₅-alkynyl radical or a C₃-C₆-cycloalkyl ring, in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl

R⁴ and R⁵ are more preferably independently of one another:

hydrogen, a C₁-C₃-alkyl radical, —NR⁸R⁹, —OR⁸, halogen, where R⁸ and R⁹ are independently of one another hydrogen, a monocyclic heteroaryl ring or a C₁-C₆-alkyl radical which are optionally substituted one or more times, identically or differently, by hydroxy, —NR¹⁰R¹¹ or —NR⁷—C(O)—R¹².

R⁴ is preferably
(i) hydrogen or
(ii) —NR⁸R⁹ or —OR⁸, where R⁸ is a C₁-C₆-alkyl radical which is substituted once by hydroxy, a —N(C₁-C₃)-alkyl or —NH—(CO)—(C₁-C₃)-alkyl radical, and R⁹ is hydrogen, or
(iii) —NR⁸R⁹, where R⁸ and R⁹ for a C₁-C₆-alkyl radical.

R⁴ is particularly preferably:

hydrogen, —NR⁸R⁹ or —OR⁸, where R⁸ is a C₁-C₆-alkyl radical which is substituted once by hydroxyl, and R⁹ is hydrogen.

R⁵ is preferably hydrogen, halogen or a C₁-C₆-alkyl radical.

It is further preferred for
(i) R⁴ and R⁵ both to be hydrogen, or
(ii) R⁴ to be —NR⁸R⁹ or —OR⁸, where R⁸ and R⁹ are independently of one another hydrogen, a monocyclic heteroaryl ring or a C₁-C₆-alkyl radical, which are optionally substituted one or more times, identically or differently, by hydroxy, —NR¹⁰R¹¹, —NR⁷—C(O)—R¹² and R⁵ is hydrogen, halogen or a C₁-C₆-alkyl radical.

It is more preferred for
(i) R⁴ and R⁵ both to be hydrogen or
(ii) R⁴ to be —NHR⁸ or —OR⁸, where R⁸ is a C₂-C₅-alkyl radical which is optionally substituted one or more times, identically or differently, by a —N(C₁-C₃)-alkyl- or —NH—(CO)—(C₁-C₃)-alkyl radical and/or by hydroxy, and R⁵ is hydrogen, halogen or a C₁-C₄-alkyl radical.

### DEFINITIONS INCLUDING ALL BUILDING BLOCKS

R⁶ in the general formula (I) may be:
(i) hydrogen or
(ii) a C₁-C₄-alkyl, C₃-C₅-alkenyl, C₃-C₅-alkynyl or C₁-C₅-alkoxy radical, a C₃-C₆-cycloalkyl or phenyl ring, a heterocyclyl ring having 3 to 6 ring atoms or a monocyclic heteroaryl ring, in each case optionally substituted one or more times, identically or differently, by hydroxy, —NR⁸R⁹, cyano, halogen, —CF₃, C₁-C₆-alkoxy and/or —OCF₃.

R⁶ is more preferably:

a C₂-C₅-alkyl, C₄-C₆-alkenyl, C₄-C₆-alkynyl or C₂-C₅-alkoxy radical, a C₄-C₆-cycloalkyl or phenyl ring, a heterocyclyl ring having 3 to 5 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R⁶ is particularly preferably:

a C₁-C₆-alkyl, a C₁-C₆-alkoxy radical or a C₃-C₇-cycloalkyl ring, in each case optionally themselves substituted one or more times, identically or differently, by hydroxy, —NR⁸R⁹ and/or C₁-C₆-alkoxy.

R⁷ in the general formula (I) may be hydrogen.

R⁸ and R⁹ in the general formula (I) may be independently of one another:

hydrogen and/or a C₁-C₅-alkyl, C₂-C₅-alkenyl radical, a C₃-C₇-cycloalkyl and/or phenyl ring and/or a monocyclic heteroaryl ring, in each case optionally substituted one or more times, identically or differently, by hydroxy, —NR¹⁰R¹¹, —NR⁷—C(O)—R¹² and/or C₁-C₆-alkoxy,

or

R⁸ and R⁹ form together with the nitrogen atom a 5- to 7-membered ring which, in addition to the nitrogen atom, optionally comprises 1 further heteroatom and which may be substituted one or more times, identically or differently, by hydroxy, —R¹⁰R¹¹ and/or C₁-C₆-alkoxy.

R⁸ and R⁹ are more preferably independently of one another: hydrogen, a monocyclic heteroaryl ring or a C₁-C₆-alkyl radical which are optionally substituted one or more times, identically or differently, by hydroxy, —NR¹⁰R¹¹, —NR⁷—C(O)—R¹².

R⁸ is particularly preferably a C₂-C₅-alkyl radical which is optionally substituted one or more times, identically or differently, by a —N(C₁-C₃)-alkyl- or —NH—(CO)—(C₁-C₃)-alkyl radical and/or by hydroxy.

R¹⁰ and R¹¹ in the general formula (I) may be independently of one another hydrogen or a C₁-C₆-alkyl radical which is optionally substituted one or more times, identically or differently, by hydroxy, halogen or C₁-C₆-alkoxy.

R¹⁰ and R¹¹ may more preferably be independently of one another hydrogen or a C₁-C₄-alkyl radical which is optionally substituted one or more times, identically or differently, by hydroxy.

R¹⁰ and R¹¹ may particularly preferably be independently of one another hydrogen or a methyl group.

R¹² in the general formula (I) may be a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring,

in each case optionally themselves substituted one or more times, identically or differently, by hydroxy, halogen, nitro, —NR⁸R⁹, C₁-C₆-alkyl and/or C₁-C₆-alkoxy.

R¹² is more preferably a C₁-C₅-alkyl, C₂-C₅-alkenyl, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy.

R¹² is particularly preferably a C₁-C₆-alkyl radical

R¹³ and R¹⁴ in the general formula (I) may preferably be independently of one another a C₁-C₆-alkyl, C₂-C₆-alkenyl and/or C₂-C₆-alkynyl radical, a C₃-C₇-cycloalkyl and/or phenyl ring, a heterocyclyl ring having 3 to 8 ring atoms and/or a monocyclic heteroaryl ring,

in each case optionally themselves substituted one or more times, identically or differently, by hydroxy, —NR⁸R⁹ and/or C₁-C₆-alkoxy.

R¹³ and R¹⁴ are more preferably independently of one another a C₁-C₅-alkyl, C₂-C₅-alkenyl and/or C₂-C₅-alkynyl radical, a C₃-C₆-cycloalkyl and/or phenyl ring, a heterocyclyl ring having 3 to 6 ring atoms and/or a monocyclic heteroaryl ring.

R¹³ and R¹⁴ are particularly preferably independently of one another a C₁-C₆-alkyl radical.

R¹⁶ in the general formula (I) may be:

a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.

R¹⁶ may more preferably be:

a C₁-C₆-alkyl radical, a C₃-C₇-cycloalkyl or phenyl ring, a heterocyclyl ring having 3 to 8 ring atoms or a monocyclic heteroaryl ring.

R¹⁶ may particularly preferably be a C₁-C₆-alkyl radical.

Likewise to be regarded as encompassed by the present invention are all compounds which result from every possible combination of the above-mentioned possible, preferred and particularly preferred meanings of the substituents.

Special embodiments of the invention moreover consist of compounds which result from combination of the meanings disclosed directly in the examples for the substituents.

A preferred subgroup of compounds of the general formula (I) with building blocks A, B, C and D are those

in which
R¹ is halogen or —CF₃,
R² is a C₁-C₁₀-alkyl radical which is optionally substituted one or more times, identically or differently, by hydroxy, —NR⁸R⁹, —NR⁷—C(O)—R¹² and/or a monocyclic heteroaryl, optionally itself substituted one or more times by a C₁-C₆-alkyl,
m is 0,
R⁴ and R⁵ are independently of one another hydrogen, —NR⁸R⁹, —OR⁸, halogen, —(CO)—NR⁸R⁹ and/or a C₁-C₆-alkyl radical,
X and Y are independently of one another —O—, —S—, —S(O)— or —NR¹⁵—, where R¹⁵ is hydrogen or a C₁-C₆-alkyl radical,
Q is a monocyclic or bicyclic heteroaryl ring,
R⁷ is hydrogen,
R⁸ and R⁹ are independently of one another hydrogen, a C₁-C₆-alkyl radical and/or a monocyclic heteroaryl ring, which are optionally substituted by hydroxy, —NR¹⁰R¹¹, —NR⁷—C(O)—R¹²,
R¹⁰ and R¹¹ are independently of one another hydrogen or a C₁-C₆-alkyl radical,
R¹² is a C₁-C₆-alkyl radical,
   and the salts, diastereomers and enantiomers thereof.

An even more preferred subgroup of compounds of the general formula (I) with building blocks A, B, C and D are those in which
R¹ is halogen or —CF₃,
R² is a C₁-C₁₀-alkyl radical which is optionally substituted one or more times, identically or differently, by hydroxy, —NR⁸R⁹, —NR⁷—C(O)—R¹² and/or a monocyclic heteroaryl, optionally itself substituted one or more times by a C₁-C₆-alkyl,
m is 0,
R⁴ and R⁵ are independently of one another hydrogen, —NR⁸R⁹, —OR⁸, halogen, —(CO)—NR⁸R⁹ and/or a C₁-C₆-alkyl radical,
X is —O—, —S—, or —NR¹⁵—;
   where R¹⁵ is hydrogen or a C₁-C₆-alkyl radical,
Y is —NR¹⁵— where R¹⁵ is hydrogen or a C₁-C₆-alkyl radical,
Q is a monocyclic or bicyclic heteroaryl ring,
R⁷ is hydrogen,
R⁸ and R⁹ are independently of one another hydrogen, a C₁-C₆-alkyl radical and/or a monocyclic heteroaryl ring, which are optionally substituted by hydroxy, —NR¹⁰R¹¹, —NR⁷—C(O)—R¹²,
R¹⁰ and R¹¹ are independently of one another hydrogen or a C₁-C₆-alkyl radical,
R¹² is a C₁-C₆-alkyl radical,
   and the salts, diastereomers and enantiomers thereof.

A very particularly preferred subgroup of compounds of the general formula (I) with building blocks A, B, C and D are those in which
R¹ is halogen,
R² is a C₁-C₁₀-alkyl radical which is optionally substituted one or more times, identically or differently, by hydroxy, —NR⁸R⁹, —NR⁷—C(O)—R¹² and/or a monocyclic heteroaryl, optionally itself substituted one or more times by C₁-C₆-alkyl,
m is 0,
R⁴ and R⁵ are independently of one another hydrogen, —NR⁸R⁹, —OR⁸, halogen, —(CO)—NR⁸R⁹ and/or a C₁-C₆-alkyl radical,
X is —O— or —NR¹⁵—,
   where R¹⁵ is hydrogen or a C₁-C₆-alkyl radical,
Y is —NR¹⁵—, where R¹⁵ is hydrogen or a C₁-C₆-alkyl radical,
Q is a monocyclic or bicyclic heteroaryl ring,
R⁷ is hydrogen,
R⁸ and R⁹ are independently of one another hydrogen, a C₁-C₆-alkyl radical and/or a monocyclic heteroaryl ring, which are optionally substituted by hydroxy, —NR¹⁰R¹¹, —NR⁷—C(O)—R¹²,
R¹⁰ and R¹¹ are independently of one another hydrogen or a C₁-C₆-alkyl radical,
R¹² is a C₁-C₆-alkyl radical,
   and the salts, diastereomers and enantiomers thereof.

### A. Preparation of the Intermediates

The group which is used in the following schemes and is designated RL is a leaving group.

Suitable leaving groups are:

—F, —Cl, —Br, —I, —OCF₃, —S—CH₃, —SOCH₃, —SO₂—CH₃, —O—SO₂—CF₃ (OTf) and other leaving groups of similar reactivity.

The individual schemes indicate the leaving groups preferred for this reaction.

### a) Preparation of the Intermediates of the Formula (II):

2,4-Dichloropyrimidines of the formula (X) can be reacted with nucleophiles of the formula (IX) to give compounds of the formula (II) (see, for example: a) U. Lücking et al, WO 2005037800; b) J. Bryant et al, WO 2004048343; c) U. Lücking et al, WO 2003076437; d) T. Brumby et al, WO 2002096888).

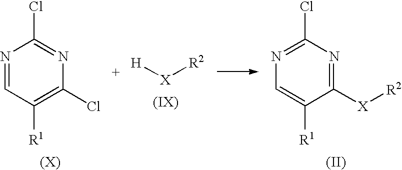

Scheme 1: Reaction of 2,4-dichloropyrimidines with nucleophiles; the substituents R¹, R² and X have the meanings indicated in general formula (I).

### Intermediate II.1:

### (2R,3R)-3-(5-Bromo-2-chloropyrimidin-4-ylamino)butan-2-ol

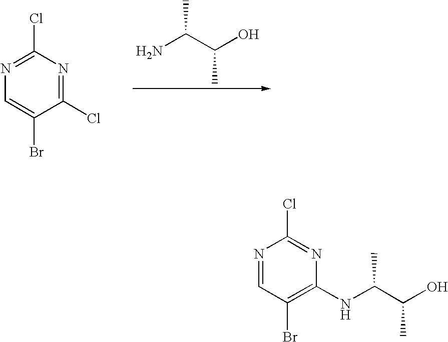

Preparation according to: Lücking et al, WO 2005/037800, page 95.

The following intermediates are prepared in analogy to intermediate 1:

| Inter- | | Analyt- | |
|---|---|---|---|
| me- | | ical | |
| diate | Structure | data | Preparation |
| II.2 | 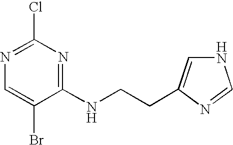 | | WO2004/048343,page 72 |
| II.3 | 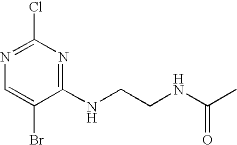 | | |

### Intermediate II.4:

### (R)-3-(5-Bromo-2-chloropyrimidin-4-ylamino)-2-methylbutan-2-ol

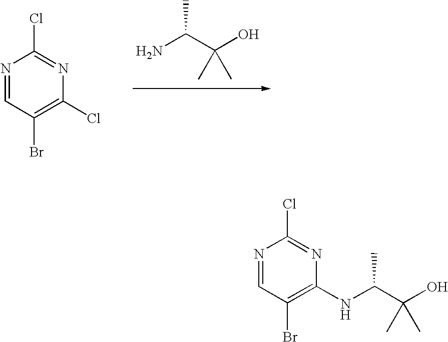

Preparation according to: Lücking et al, WO 2005/037800, page 94.

### Intermediate II.5:

### (2R,3R)-3-(2-Chloro-5-trifluoromethylpyrimidin-4-ylamino)butan-2-ol

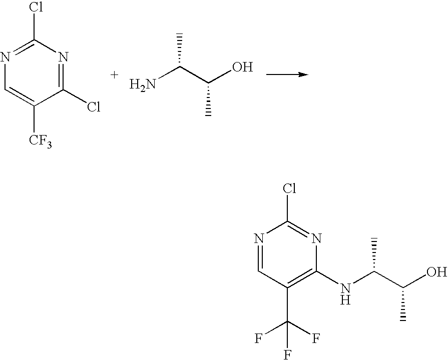

4.83 ml (34.8 mmol) of triethylamine are added dropwise to a reaction mixture of 3.78 g (17.4 mmol) of 2,4-dichloro-5-trifluoromethylpyrimidine (Frontier Scientific) and 2.19 g (17.4 mmol) of (2R,3R)-3-aminobutan-2-ol hydrochloride in 70 ml of acetonitrile at 0° C. The ice bath is removed and the mixture is stirred at room temperature for 48 hours. The mixture is added to half-concentrated NaCl solution and extracted with ethyl acetate (2×). The combined organic phases are dried (Na₂SO₄), filtered and concentrated. The resulting residue is purified by preparative HPLC. 1.45 g (5.36 mmol; 31% yield) of the product are obtained.

Column: XBridge C18 5μ 100×30 mm

Eluents: A:H2O B:acetonitrile

Buffer: A/0.1% TFA

Gradient: 60% A+40% B(2′)_{—}40->70% B(10′)->99% B(0.5′)

Flow rate: 40.0 mL/min

Detection: DAD (210-500 nm) TAC; MS-ESI+(125-800 m/z) TIC

Temperature: Rt

Retention: 5.0-6.0 min

¹H-NMR (DMSO): 8.38 (s, 1H), 6.72 (d, 1H), 5.00 (d, 1H), 4.09 (m, 1H), 3.71 (m, 1H), 1.12 (d, 3H), 1.01 (d, 3H).

### Intermediate II.6:

### 5-Bromo-2-chloro-4-methylsulphanylpyrimidine

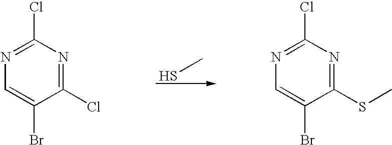

2 g of sodium methanethiolate (30 mmol) and 6.5 g of 5-bromo-2,4-dichloropyrimidine (28.5 mmol) were stirred in 50 mL of dry acetonitrile at RT for 24 h. The mixture was added to ice-water, extracted 3× with dichloromethane, dried with sodium sulphate and concentrated. The product was recrystallized from hexane.

Yield 4 g (70%) of 5-bromo-2-chloro-4-methylsulphanylpyrimidine

MS (ES+) 241

### Intermediate II.7:

### (2R,3R)-3-(5-Bromo-2-chloropyrimidin-4-yloxy)butan-2-ol

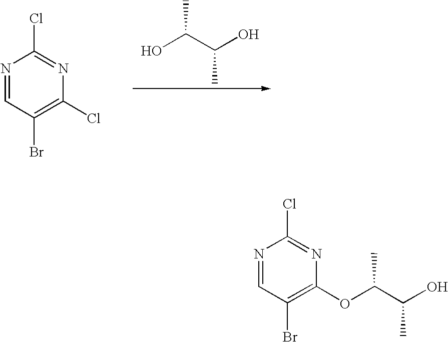

Preparation according to: Lücking et al, WO 2005/037800, page 93.

### Intermediate II.8

### 2-(2-Chloropyrimidin-4-ylamino)ethanol

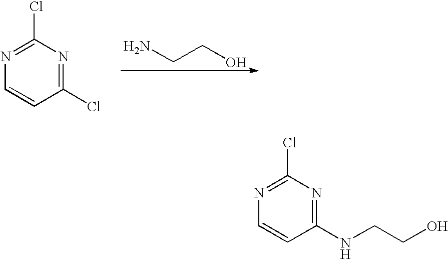

0.93 ml (6.7 mmol) of triethylamine was added to a suspension of 1 g (6.7 mmol) of 2,4-dichloropyrimidine in 15 ml of 2:1 acetonitrile/DMF while cooling in an ice bath, and a solution of 0.4 ml (6.7 mmol) of 2-aminoethanol in 1 ml of acetonitrile was added dropwise. The mixture was then stirred at room temperature for 12 h. The reaction mixture was filtered and thoroughly washed with dichloromethane, and the mother liquor was concentrated. Chromatography (hexane/ethyl acetate 1:1->DCM/MeOH 4:1) afforded 148 mg (13% of theory) of intermediate II.12

¹H-NMR (DMSO):7.92 (d, 1H), 7.83 (d, 1H), 6.44 (m, 1H), 4.76 (d, 1H), 3.46 (m, 2H), 3.31 (m, 2H)

MS: 174 (ES).

### b) Intermediates of the Formula (III):

Intermediates of the formula (III), especially of the formula (IIIa), (IIIb), (IIIc) and (IIId), are to a large extent commercially available or can be prepared by known methods.

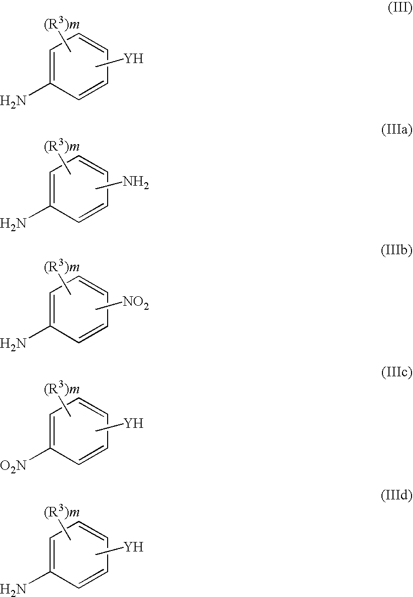

### a) Intermediates of the Formula (V):

Intermediates of the formula (V), in particular of the formula (Va), (Vb) and (Vc), are to a large extent commercially available or can be prepared by various known methods.

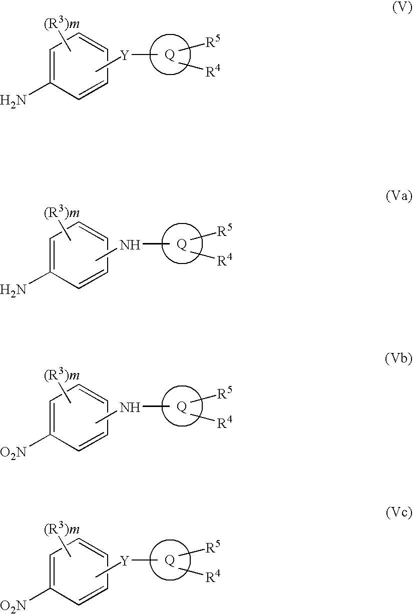

The substituents Q, R³, R⁴, R⁵, Y and m have the meanings in general formula (I).

The following intermediates are commercially available for example.

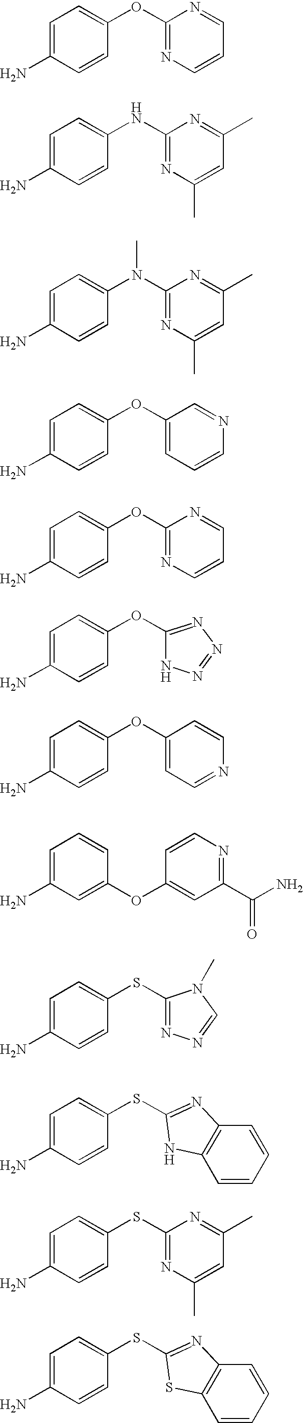

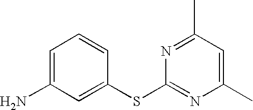

### Synthesis of Intermediates of the Formula (V)

### Process Variant A I

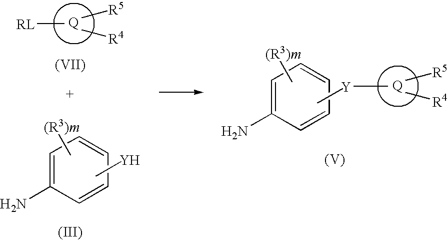 [0281] Scheme 2: Direct reaction of aniline derivatives of the formula (III) with electrophiles of the formula (VII); the substituents Q, R³, R⁴, R⁵ and m have the meanings indicated in general formula (I).
   RL as leaving group is in particular Br, Cl, I and OTf.

### Process Variant A II

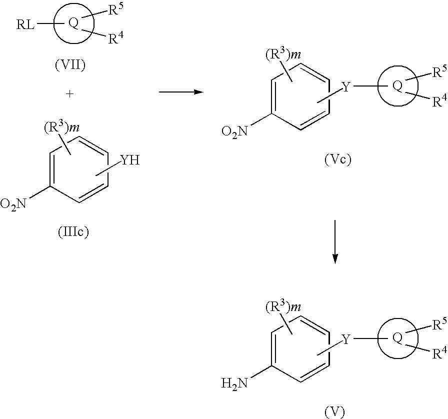 [0284] Scheme 3: Reaction of nitroanilines of the formula (IIIc) with electrophiles of the formula (VII) and subsequent reduction of the nitro group; the substituents Q, R³, R⁴, R⁵ and m have the meanings indicated in general formula (I).
   RL as leaving group is in particular Br, Cl, I and OTf.

### Intermediate V.1 (by Process Variant A I)

### N-Pyridin-2-ylbenzene-1,4-diamine

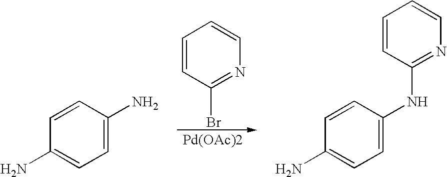

A suspension of 2.7 g (25 mmol) of 1,4-phenylenediamine, 0.48 ml (5 mmol) of 2-bromopyridine, 13.82 g (100 mmol) of potassium carbonate, 22.5 mg (0.1 mmol) of Pd(II) acetate and 62.5 mg (0.1 mmol) of rac-BINAP are boiled under reflux in 50 ml of toluene under an N2 atmosphere for 8 h. This is followed by filtration with suction, substantial concentration of the mother liquor, removal of crystals which have separated out (filtered off with suction; precursor diamine) and reconcentration. Chromatography (silica gel; hexane/ethyl acetate 1:1) of the mother liquor results in 620 mg (67% of theory) of intermediate V.1. N-Pyridin-2-ylbenzene-1,4-diamine.

¹H-NMR (DMSO): 8.32 (s, 1H), 7.98 (m, 1H), 7.38 (m, 1H), 7.15 (m, 2H), 6.52 (m, 4H), 4.69 (s, 2H)

MS: 186 (ES).

### Intermediate V.1 (by Process Variant A II):

### N-Pyrimidin-2-ylbenzene-1,4-diamine

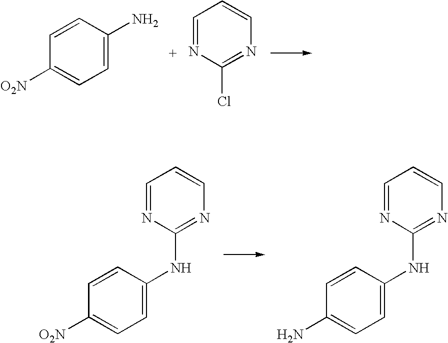

5 ml (20 mmol) of 4 molar HCl in dioxane and 5 ml of water are added to a solution of 2.76 g (20 mmol) of 4-nitroaniline and 2.29 g (20 mmol) of 2-chloropyrimidine in 150 ml of acetonitrile, and the mixture is boiled under reflux for 12 h. After cooling, 3.33 ml (24 mmol) of triethylamine are added, and the crystals are filtered off with suction, washed with acetonitrile and water and then dried in vacuo at +60° C. 1.07 g (25% of theory) of 4-nitrophenyl)pyrimidin-2-ylamine are obtained in this way.

¹H-NMR (DMSO): 10.45 (s, 1H), 8.59 (d, 2H), 8.17 (m, 2H), 7.99 (m, 2H), 7.0 (m, 1H)

MS: 217 (ES).

A solution of 1.07 g (4.95 mmol) of (4-nitrophenyl)pyrimidin-2-ylamine in 80 ml of 1:1 THF/ETOH is hydrogenated with palladium on carbon (10%) under a hydrogen atmosphere at room temperature. Removal of catalyst by filtration and concentration result in 0.88 g (95% of theory) of intermediate V.1.

¹H-NMR (DMSO): 9.0 (s, 1H), 8.30 (d, 2H), 7.27 (m, 2H), 6.62 (m, 1H), 6.46 (m, 2H) 4.70 (m, 2H)

MS: 187 (ES).

### d) Intermediates of the Formula (VI):

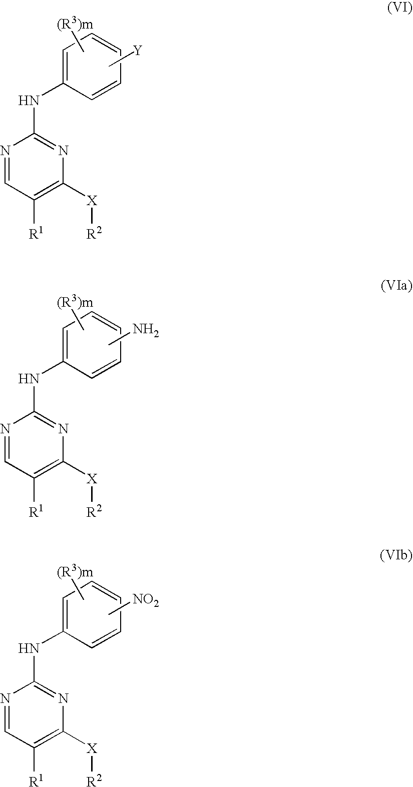

### Process Variant A III

2-Chloropyrimidines of the formula (II) can be reacted with nucleophiles of the formula (IIId) to give compounds of the formula (VI)

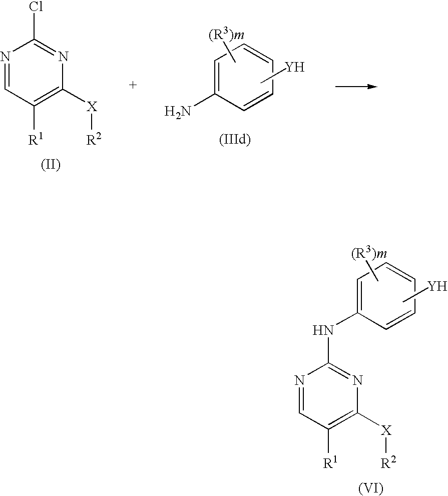

Scheme 4: Preparation of intermediates of the type (VI); the substituents R¹, R², R³, Y and m have the meanings indicated in general formula (I).

### Process Variant A IV

Alternatively, 2-chloropyrimidines of the formula (II) are reacted first with nitroanilines of the formula (IIIc) to give compounds (VIb) in which the nitro group is then in turn reduced to result in intermediates of the formula (VIa). Any number of methods are available for reducing the nitro group (see, for example: R. C. Larock, Comprehensive Organic Transformations, VCH, New York, 1989, 411-415). For example, the described hydrogenation using Raney nickel or the use of titanium(III) chloride in THF is suitable

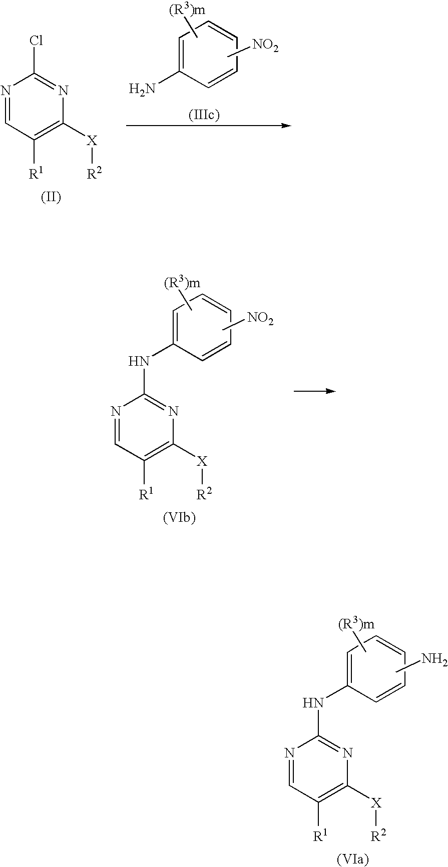

Scheme 5: Preparation of intermediates of type (VI) via nitro intermediate. The substituents R¹, R², R³ and m have the meanings indicated in general formula (I).

### Process Variant A V

The mother liquor of the reaction of process variant B I usually contains intermediates of the formula (VI), so that they can be obtained by concentration and purification of the mother liquor.

### Intermediate VI.1

### (2R,3R)-3-[2-(4-Aminophenylamino)-5-bromopyrimidin-4-ylamino]butan-2-ol

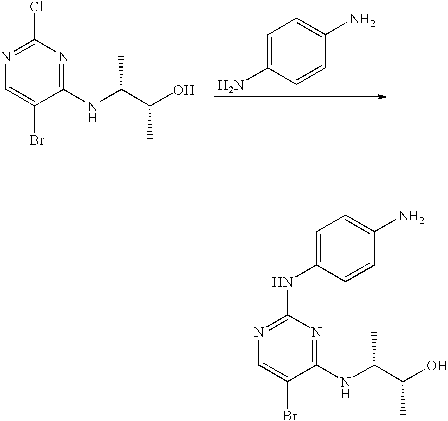

The mother liquor from the preparation of Example 1 is concentrated and purified by chromatography (DCM/MeOH 9:1). 73 mg of intermediate VI.1 are obtained in this way.

¹H-NMR (DMSO): 8.67 (s, 1H), 7.88 (s, 1H), 7.23 (d 2H), 6.43 (d, 2H), 5.82 (d, 1H), 4.91 (d, 1H), 4.66 (s, 2H), 3.97 (m, 1H), 3.71 (m, 1H), 1.12 (d, 3H), 1.02 (d, 3H)

MS: 352 (ES).

### Intermediates VI.2 and VI.3:

### Intermediates VI.2

### (2R,3R)-3-[5-Bromo-2-(4-nitrophenylamino)pyrimidin-4-yloxy]butan-2-ol

and

### Intermediate VI.3

### (2R,3R)-3-[2-(4-aminophenylamino)-5-bromopyrimidin-4-yloxy]butan-2-ol

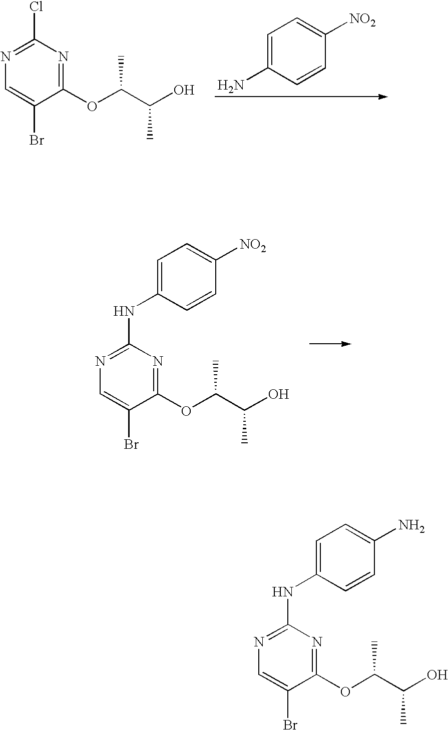

1 ml of water and 1 ml of 4 molar hydrochloric acid in dioxiane are added to a solution of 1.126 g (4 mmol) of (2R,3R)-3-(5-bromo-2-chloropyrimidin-4-yloxy)butan-2-ol and 0.552 g (4 mmol) of 4-nitroaniline in 20 ml of acetonitrile, and the mixture is then stirred at +95° C. for 16 h. After cooling, 1 equivalent of triethylamine is added and the crystals which have separated out are filtered off with suction. 990 mg 64.5% of theory of (2R,3R)-3-[5-bromo-2-(4-nitrophenylamino)pyrimidin-4-yloxy]butan-2-ol (intermediate VI.2) are obtained in this way.

¹H-NMR (DMSO): 10.41 (s, 1H), 8.46 (s, 1H), 8.16 (d, 2H), 7.91 (d, 2H), 5.19 (m, 1H), 4.87 (m, 1H), 3.79 (m, 1H), 1.26 (d, 3H), 1.10 (d, 3H)

MS: 383 (ES).

56.59 g (25.7 mmol) of iron(III) sulphate are dissolved in 190 ml of water, and 59 ml of 25% strength aqueous ammonia are added. Then a suspension of 7.8 g (20.35 mmol) of (2R,3R)-3-[5-bromo-2-(4-nitrophenylamino)pyrimidin-4-yloxy]butan-2-ol (compound 4.2) in 234 ml of methanol is added dropwise at RT. The mixture is then stirred at +90° C. for 3 h. After cooling, it is filtered through Celite and thoroughly washed with MeOH, and the mother liquor is concentrated and purified by chromatography (dichloromethane/MeOH 4:1). 5.68 g (79% of theory) of intermediate VI.3 are obtained in this way.

¹H-NMR (DMSO): 9.11 (s, 1H), 8.17 (s, 1H), 7.21 (d, 2H), 6.47 (d, 2H), 5.1 (m, 1H), 4.78 (m, 3H), 3.75 (m, 1H), 1.18 (d, 3H), 1.06 (d, 3H)

MS: 353 (ES).

The following intermediates were prepared in analogy to intermediate VI.2 and VI.3:

| Intermediate | Structure | Analysis |
|---|---|---|
| VI.4 | 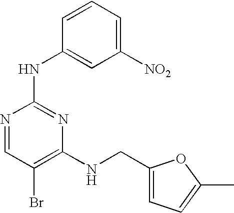 | MS: 404(ES+) |
| VI.5 | 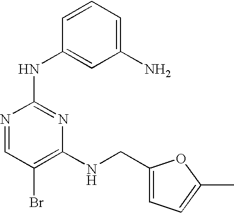 | MS: 374(ES+) |

### e) Intermediates of the Formula (VII):

Intermediates of the formula (VII) are to a large extent commercially available or can be prepared by various known methods.

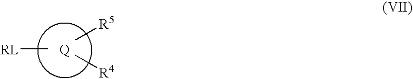

Substituents Q, R⁴ and R⁵ have the meanings indicated in general formula (I). RL as leaving group is in particular —Cl, —Br, —I or —OTf.

The following intermediates of the formula VII are commercially available for example:

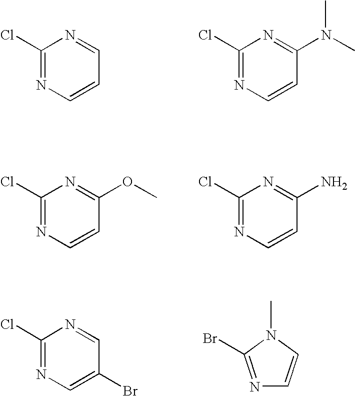

### Synthesis of Intermediates of the Formula VII:

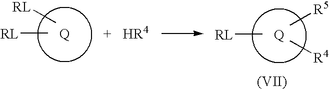

For example, 4-dichloropyrimidines of the formula (X) can be reacted with nucleophiles of the formula (IX) to give compounds of the formula (II). Compounds of the formula (II) are a subgroup of intermediates of the formula VII with Q equal to pyrimidine, RL equal to Cl, R⁴ equal to XR² and R⁵ equal to R¹.

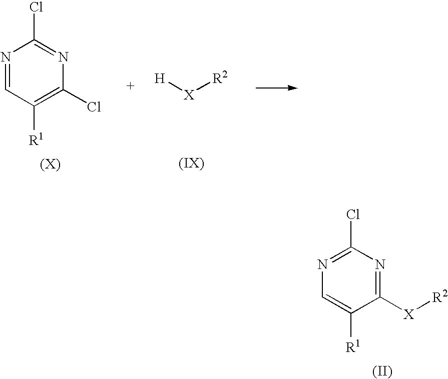

Scheme 6: Reaction of dichloroanilines of the formula (X) with nucleophiles of the formula (IX). The substituents X, R¹ and R² have the meanings indicated in general formula (I).

### Intermediate VII.1:

### 2-(2-Chloropyrimidin-4-ylamino)ethanol

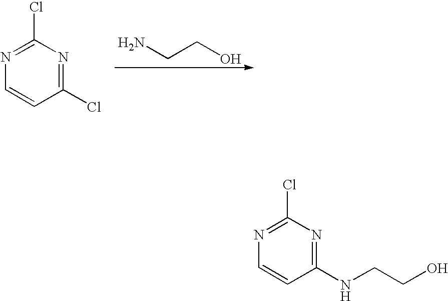

0.93 ml (6.7 mmol) of triethylamine is added to a suspension of 1 g (6.7 mmol) of 2,4-dichloropyrimidine in 15 ml of 2:1 acetonitrile/DMF while cooling in an ice bath, and a solution of 0.4 ml (6.7 mmol) of 2-aminoethanol in 1 ml of acetonitrile is added dropwise. The mixture is then stirred at room temperature for 12 h. The reaction mixture is filtered and thoroughly washed with dichloromethane, and the mother liquor concentrates. Chromatography (hexane/ethyl acetate 1:1->DCM/MeOH 4:1) afforded 148 mg (13% of theory) of intermediate VII.1.

¹H-NMR (DMSO):7.92 (d, 1H), 7.83 (d, 1H), 6.44 (m, 1H), 4.76 (d, 1H), 3.46 (m, 2H), 3.31 (m, 2H)

MS: 174 (ES).

The following intermediates are prepared in analogy to intermediate VII.1:

| Intermediate | Structure | Analysis |
|---|---|---|
| VII.2 | 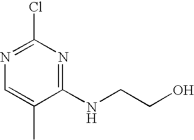 | MS: 188(ES+) |
| VII.3 | 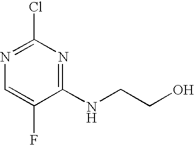 | MS: 192(ES+) |
| VII.4 | 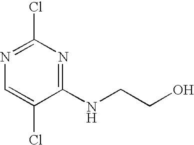 | MS: 208(ES+) |
| VII.5 | 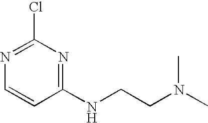 | MS: 201(ES+) |

### Intermediate VII.6:

### 2-(2-Chloropyrimidin-4-yloxy)ethanol

and

### Intermediate VII.7:

### 2-(4-chloropyrimidin-2-yloxy)ethanol

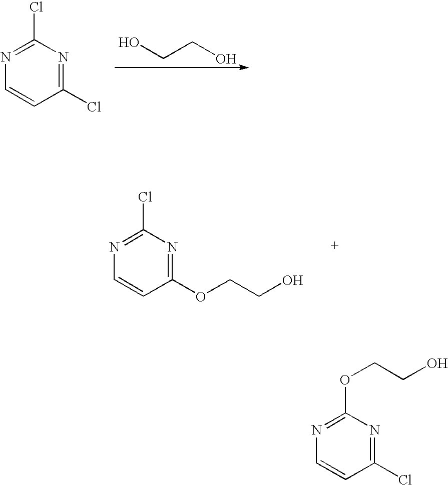

327 mg (7.5 mmol) of sodium hydride (55-60% in oil) are added in portions to a solution of 0.42 ml (7.5 mmol) of ethylene glycol in 10 ml of 1:1 acetonitrile/NMP at room temperature, and the mixture is then stirred at RT for a further 15 min. The resulting suspension is added in portions to a solution of 1.34 g (9 mmol) of 2,4-dichloropyrimidine in 10 ml of acetonitrile while cooling in an ice bath. The mixture is then allowed slowly to reach room temperature and is stirred for 12 h. The mixture is then poured into ice-water, extracted with ethyl acetate, dried with sodium sulphate, concentrated and purified by chromatography (silica gel; gradient hexane/ethyl acetate 2:1->pure ethyl acetate). 275 mg (21% of theory) of a 4:1 mixture of regioisomers (according to H-NMR) of 2-(2-chloropyrimidin-4-yloxy)ethanol and 2-(4-chloropyrimidin-2-yloxy)ethanol are obtained in this way.

MS: 175 (ES+)

The following intermediate is prepared in analogy to intermediate VII.7:

| Inter- | | | |
|---|---|---|---|
| mediate | Structure | Analysis | Remarks |
| VII.8 | 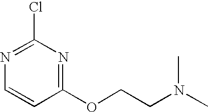 | MS: 202(ES+) | Regioselectivereaction |

### f) Intermediates of the Formula (VIII):

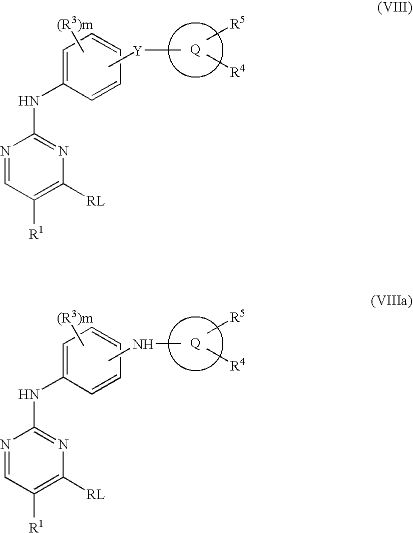

Substituted 2-chloropyrimdines of the formula (XI) can be reacted with nucleophiles of the formula (V) to give compounds of the formula (VIII).

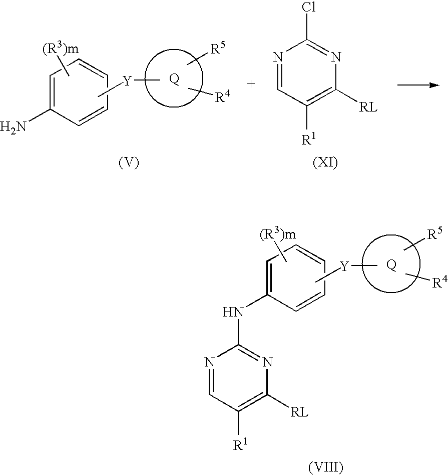

Scheme 7: The substituents Q, R¹, R², R³, R⁴, R⁵, X, Y and m have the meanings indicated in general formula (I).
   RL as masked leaving group is in particular SMe and S-alkyl.

### Intermediate VIII.1

### N-(5-Bromo-4-methylsulphanylpyrimidin-2-yl)-N′-pyrimidin-2-yl-benzene-1,4-diamine

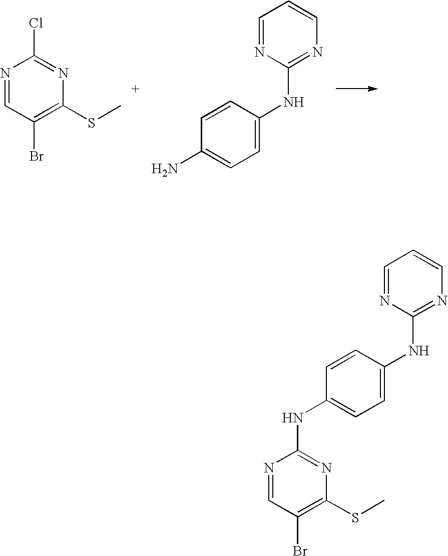

A solution of 782 mg (3.26 mmol) of N-pyrimidin-2-ylbenzene-1,4-diamine and 608 mg (3.26 mmol) of 5-bromo-2-chloro-4-methylsulphanylpyrimidine in 13.3 ml of 10:1 n-butanol/MeOH is stirred at +60° C. for 40 hours. It is then concentrated, acetonitrile is added, and the crystals are filtered off with suction. The crystals are washed with acetonitrile and water and then dried at +40° C. in vacuo. 320 mg (25% of theory) of intermediate VIII.1 are obtained in this way.

¹H-NMR (DMSO): 9.59 (s, 1H), 9.46 (s, 1H), 8.40 (d, 2H), 8.21 (s, 1H), 7.58 (m, 4H), 6.75 (m, 1H), 2.51 (s, 3H)

ES+: 391

The following intermediate is prepared in analogy to intermediate VIII.1:

| Intermediate | Structure | Analysis |
|---|---|---|
| VIII.2 | 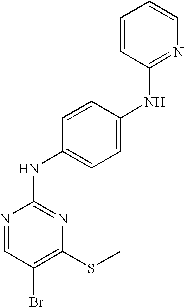 | MS: 390(ES+) |

The intermediates of the formula VIII may, depending on the masked leaving group, already be compounds which are covered by general formula (I) and show activity as protein kinase inhibitors, including intermediates VIII.1 and intermediate VIII.2 (compounds 23 and 29).

### B. Preparation of the Compounds According to the Invention

### Process Variant B I:

2-Chloropyrimidines of the formula (II) can be reacted with phenylenediamines of the formula (IIIa) to give the desired symmetrical target compounds of the formula (IV).

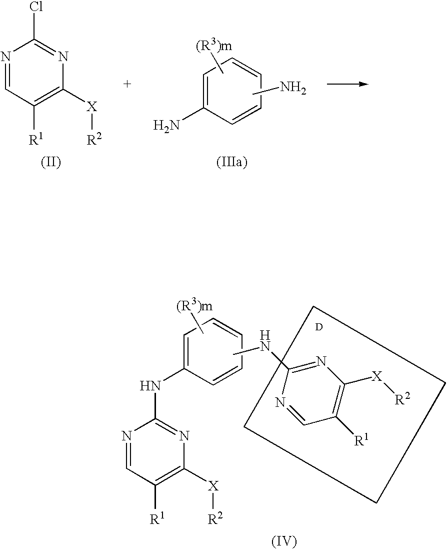

Scheme 8: General approach to symmetrical target compounds The substituents R¹, R², R³, X and m have the meanings indicated in general formula (I), and in building block D of the formula (IV) X—R² has the meaning of R⁴ and R¹ has the meaning of R⁵.

### EXAMPLE 1

### (2R,3R)-3-(5-Bromo-2-{4-[5-bromo-4-((1R,2R)-2-hydroxy-1-methyl propylamino)-pyrimidin-2-ylamino]phenylamino}pyrimidin-4-ylamino)butan-2-ol

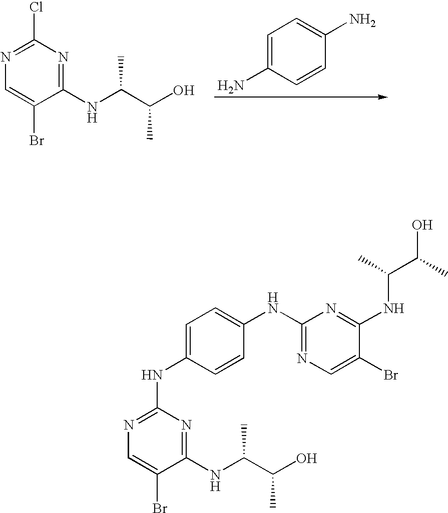

2 equivalents of 4 molar hydrochloric acid in dioxane are added to a solution of 281 mg (1 mmol) of (2R,3R)-3-(5-bromo-2-chloropyrimidin-4-ylamino)butan-2-ol and 216 mg (2 mmol) of 1,4-phenylenediamine in 4 ml of acetonitrile and 0.6 ml of water, and the mixture is stirred at +95° C. for 12 h. After cooling, the crystals are filtered off with suction, washed with acetonitrile and dried in vacuo at +60° C. 38 mg (6% of theory) of compound 1 are obtained.

¹H-NMR (DMSO): 9.03 (s, 2H), 7.96 (s, 2H), 7.52 (m, 4H), 5.91 (d, 2H), 4.96 (d, 2H), 4.01 (m, 2H), 3.71 (m, 2H), 1.15 (d, 6H), 1.04 (d, 6H),

MS: 597 (ES).

The mother liquor contains intermediate 9 (see section B. IV Preparation of the intermediates of the formula VI).

### EXAMPLE 2

### R)-3-(5-Bromo-2-{4-[5-bromo-4-((R)-2-hydroxy-1,2-dimethylpropylamino)-pyrimidin-2-ylamino]phenylamino}pyrimidin-4-ylamino)-2-methylbutan-2-ol

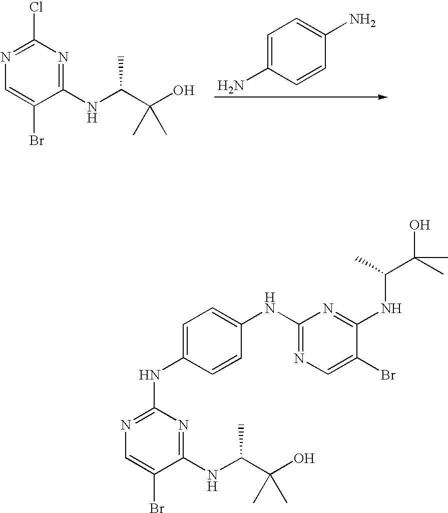

0.75 ml (3 mmol) of 4 molar hydrochloric acid in dioxane is added to a solution of 884 mg (3 mmol) of (R)-3-(5-bromo-2-chloropyrimidin-4-ylamino)-2-methylbutan-2-ol and 649 mg (6 mmol) of 1,4-phenylenediamine in 50 ml of acetonitrile, and the mixture is stirred at +95° C. for 16 h. After cooling, the crystals are filtered off with suction, washed with acetonitrile and dried in vacuo at +60° C. 95 mg (5% of theory) of compound 2 are obtained in this way.

¹H-NMR (DMSO): 9.07 (s, 2H), 8.01 (s, 2H), 7.56 (s, 4H), 5.95 (s, 2H), 4.82 (s, 2H), 4.1 (m, 2H), 1.18 (d, 9H), 1.13 (d, 9H)

MS: 625 (ES).

### EXAMPLE 3

### (R)-3-(5-Bromo-2-{3-[5-bromo-4-((R)-2-hydroxy-1,2-dimethyl propylamino)-pyrimidin-2-ylamino]phenylamino}pyrimidin-4-ylamino)-2-methylbutan-2-ol

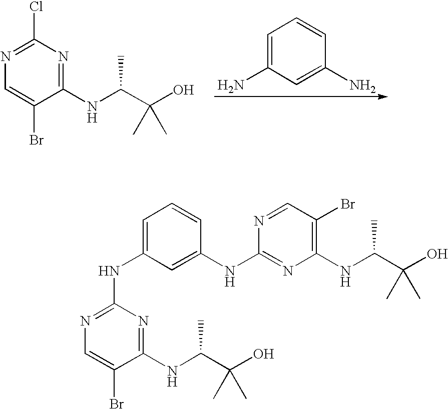

4 molar hydrochloric acid in dioxane is added to a solution of 295 mg (1 mmol) of (R)-3-(5-bromo-2-chloropyrimidin-4-ylamino)-2-methylbutan-2-ol and 216 mg (2 mmol) of 1,3-phenylenediamine hydrochloride in 7.5 ml of acetonitrile and 0.6 ml of water, and the mixture is stirred at +95° C. for 16 h. After cooling, the crystals are filtered off with suction, washed with acetonitrile and dried in vacuo at +60° C. 157 mg (43% of theory) of compound 3 are obtained in this way.

¹H-NMR (DMSO): 10.12 (s, 2H) 8.21 (s, 2H), 8.1 (s, 1H), 7.28 (m, 3H), 6.82 (s, 2H), 4.0 (m, 1H), 1.03 (m, 18H)

MS: 625 (ES)

### Process Variant B II:

2-Chloropyrimidines of the formula (II) can be reacted with various substituted anilines of the formula (V) to give the desired target compounds of the formula (I).

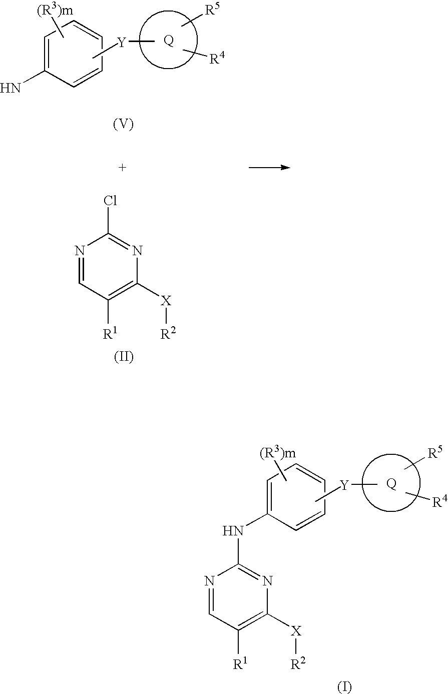

Scheme 9: General approach to nonsymmetrical target compounds; the substituents Q, R¹, R², R³, R⁴, R⁵, X, Y and m have the meanings indicated in general formula (I).

### EXAMPLE 4

### (2R,3R)-3-{2-[4-(Pyrimidin-2-yloxy)phenylamino]-5-trifluoromethylpyrimidin-4-ylamino}butan-2-ol

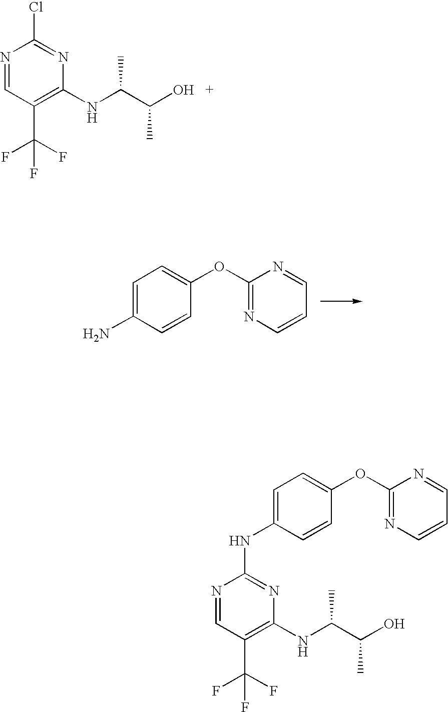

0.3 equivalents of 4 molar hydrochloric acid in dioxane is added to a solution of 37 mg (0.2 mmol) of 4-(2-pyrimidinyloxy)phenylamine and 54 mg (0.2 mmol) of (2R,3R)-3-(2-chloro-5-trifluoromethylpyrimidin-4-ylamino)butan-2-ol in 4.4 ml of 10:1 n-butanol/MeOH, and the mixture is stirred at +60° C. for 24 hours. After cooling, 1.3 equivalents (0.036 ml) of triethylamine are added, and the mixture is concentrated and then flash chromatographed on silica gel with DCM/MeOH 9:1. 45 mg (53% of theory) of compound 4 are obtained in this way.

¹H-NMR (DMSO): 9.67 (s, 1H), 8.59 (d, 2H), 8.19 (s, 1H), 7.74 (d, 2H), 7.20 (m, 1H), 7.06 (m, 2H), 5.94 (d, 1H), 5.04 (d, 1H), 4.1 (m, 1H), 3.73 (m, 1H), 1.16 (d, 3H), 1.04 (d, 3H)

MS: 421 (ES).

### Process Variant B III

Substituted anilinopyrimdines of the formula (VI) can be reacted with electrophiles of the formula (VII) to give compounds of the formula (I). This reaction can be catalysed both by acids, bases or metals (e.g. palladium complexes or copper complexes).

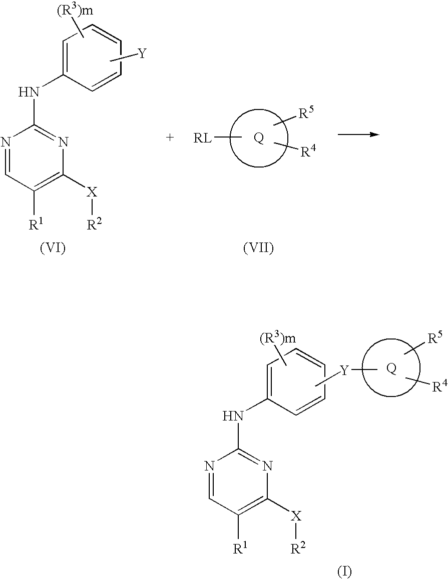

Scheme 10: Preparation of the target compounds by direct addition of heteroaromatic compounds onto anilinopyrimdines. The substituents Q, R¹, R², R³, R⁴, R⁵, X, Y and m have the meanings indicated in general formula (I).
   RL is a leaving group such as, for example, Cl, F, Br, I, OTf.

### EXAMPLE 5

### (2R,3R)-3-{5-Bromo-2-[4-(pyrimidin-2-ylamino)phenylamino]pyrimidin-4-ylamino}butan-2-ol

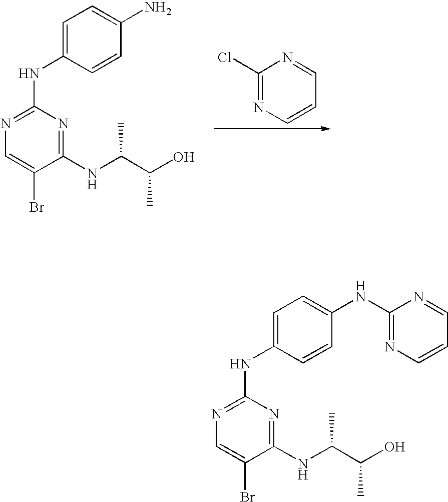

1.5 equivalents 4 molar hydrochloric acid in dioxane are added to a solution of 71 mg (0.2 mmol) of (2R,3R)-3-[2-(4-aminophenylamino)-5-bromopyrimidin-4-ylamino]butan-2-ol and 23 mg (0.2 mmol) of 2-chloropyrimidine in 3.3 ml of 10:1 n-butanol/MeOH, and the mixture is stirred at +60° C. for 16 hours. After cooling, 2 equivalents of triethylamine are added, and the mixture is concentrated and purified by chromatography (DCM/MeOH 9:1). 15 mg (18% of theory) of compound are obtained in this way.

¹H-NMR (DMSO): 9.37 (s, 1H), 9.04 (s, 1H), 8.38 (m, 2H), 7.96 (s1H), 7.54 (s, 4H), 6.71 (m, 1H), 5.91 (d, 1H), 4.95 (d, 1H), 3.74 (m, 1H), 3.72 (m, 1H), 1.15 (d, 3H), 1.05 (d, 3H)

MS: 430 (ES).

### EXAMPLE 6

### (2R,3R)-3-(5-Bromo-2-{4-[4-(2-hydroxyethylamino)pyrimidin-2-ylamino]-phenylamino}pyrimidin-4-ylamino)butan-2-ol

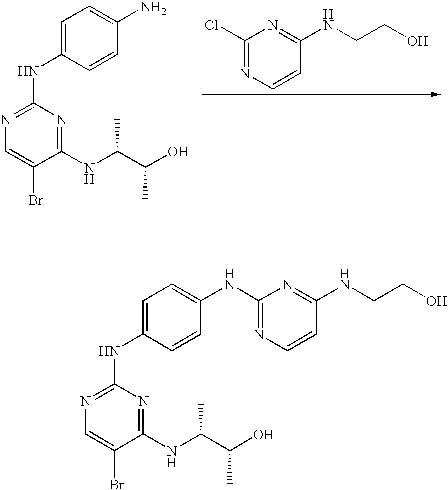

0.3 equivalents of 4 molar hydrochloric acid in dioxane is added to a solution of 71 mg (0.2 mmol) of (2R,3R)-3-[2-(4-aminophenylamino)-5-bromopyrimidin-4-ylamino]butan-2-ol and 35 mg (0.2 mmol) of 2-(2-chloropyrimidin-4-ylamino)ethanol in 3.3 ml of 10:1 n-butanol/MeOH, and the mixture is stirred at +60° C. for 16 hours. After cooling, 1.3 equivalents (0,036 ml) of triethylamine are added, and the mixture is concentrated. It is then taken up in acetonitrile, and the crystals are filtered off with suction and washed with a little acetonitrile and water. Drying in vacuo at +40° C. results in 73 mg (75% of theory) of compound 6.

¹H-NMR (DMSO): 10.16 (s, 1H), 9.31 (s, 1H), 8.94 (s, 1H), 8.01 (s, 1H), 7.70 (m, 3H), 7.36 (m, 2H), 6.18 (d, 1H), 6.02 (d, 1H), 4.97 (m, 2H), 4.04 (m, 2H), 3.42 (m, 2H), 1.16 (d, 3H), 1.04 (d, 3H)

MS: 489 (ES).

### EXAMPLE 7

### (2R,3R)-3-(5-Bromo-2-{4-[4-(2-hydroxyethylamino)pyrimidin-2-ylamino]-phenylamino}pyrimidin-4-yloxy)butan-2-ol

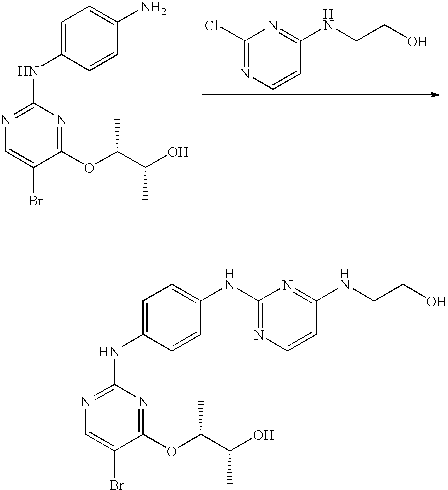

0.3 equivalents of 4 molar hydrochloric acid in dioxane is added to a solution of 71 mg (0.2 mmol) of (2R,3R)-3-[2-(4-aminophenylamino)-5-bromopyrimidin-4-yloxy]butan-2-ol and 35 mg (0.2 mmol) of 2-(2-chloropyrimidin-4-ylamino)ethanol in 3.3 ml of 10:1 n-butanol/MeOH, and the mixture is stirred at +60° C. for 16 hours. After cooling, 1.3 equivalents (0.036 ml) of triethylamine are added, and the mixture is concentrated. It is then taken up in acetonitrile, and the crystals are filtered off with suction and washed with a little acetonitrile and water. Drying in vacuo at +40° C. results in 60 mg (61% of theory) of compound 7.

¹H-NMR (DMSO): 10.3 (s, 1H), 9.72 (s, 1H), 9.02 (s, 1H), 8.32 (s, 1H), 7.68 (m, 3H), 7.41 (m, 2H), 6.20 (d, 1H), 5.15 (m, 1H), 4.87 (m, 2H), 3.78 (m, 1H), 3.54 (m, 2H), 3.42 (m, 2H), 1.23 (d, 3H), 1.08 (d, 3H)

MS: 490 (ES).

### EXAMPLES 8 AND 9

### EXAMPLE 8

### (2R,3R)-3-(5-Bromo-2-{4-[4-(2-hydroxyethoxy)pyrimidin-2-ylamino]-phenylamino}pyrimidin-4-yloxy)butan-2-ol

and

### EXAMPLE 9

### (2R,3R)-3-(5-bromo-2-{4-[2-(2-hydroxyethoxy)pyrimidin-4-ylamino]-phenylamino}pyrimidin-4-yloxy)butan-2-ol

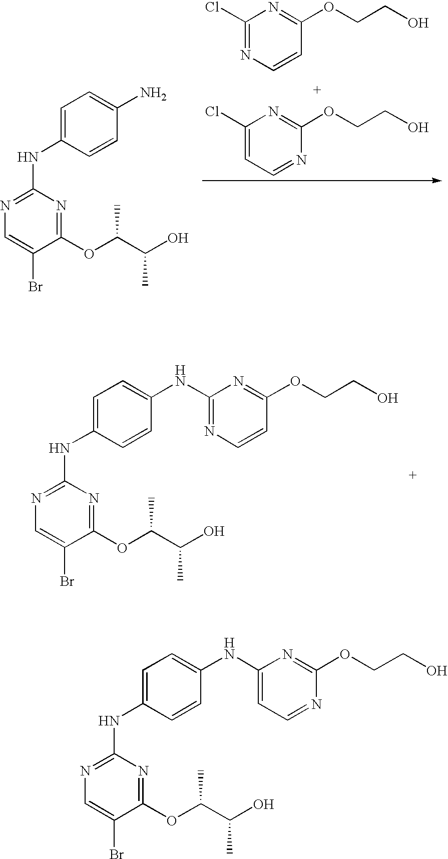

0.3 equivalents of 4 molar hydrochloric acid in dioxane is added to a solution of the mixture of regioisomers of 2-(2-chloropyrimidin-4-yloxy)ethanol and 2-(4-chloro-pyrimidin-2-yloxy)ethanol (35 mg, 0.2 mmol) and 71 mg (0.2 mmol) of (2R,3R)-3-[2-(4-aminophenylamino)-5-bromopyrimidin-4-yloxy]butan-2-ol in 4.4 ml of 10:1 n-butanol/MeOH, and the mixture is stirred at +60° C. for 24 hours. After cooling, 1.3 equivalents (0.036 ml) of triethylamine are added, and the mixture is concentrated and then flash chromatographed on silica gel with DCM/MeOH 9:1. 22 mg (22% of theory) of compound 8 and 15 mg (15% of theory) of compound are obtained in this way.

Compound 8:

¹H-NMR (DMSO): 9.56 (s, 1H), 9.45 (s, 1H), 8.28 (s, 1H), 7.97 (d, 1H), 7.58 (m, 2H), 7.52 (m, 2H), 6.33 (d, 1H), 5.15 (m, 1H), 4.82 (m, 2H, 4.20 (m, 2H), 3.77 (m 1H), 3.66 (m, 2H), 1.22 (d, 3H), 1.08 (d, 3H)

MS: 493 (ES).

Compound 9:

¹H-NMR (DMSO): 9.49 (s, 1H), 9.34 (s, 1H), 8.26 (s, 1H), 8.13 (d, 1H), 7.59 (m, 2H), 7.54 (m, 2H), 6.18 (d, 1H), 5.13 (m, 1H), 4.83 (m, 2H), 4.28 (m, 2H), 3.77 (m 1H), 3.68 (m, 2H), 1.20 (d, 3H), 1.08 (d, 3H)

MS: 493 (ES).

### EXAMPLES 10 AND 11

### EXAMPLE 10

### (2R,3R)-3-(5-Bromo-2-{4-[4-(2-dimethylaminoethoxy)pyrimidin-2-ylamino]-phenylamino}pyrimidin-4-yloxy)butan-2-ol

and

### EXAMPLE 11

### 2-{4-[5-bromo-4-((1R,2R)-2-hydroxy-1-methylpropoxy)pyrimidin-2-ylamino]-phenylamino}pyrimidin-4-ol

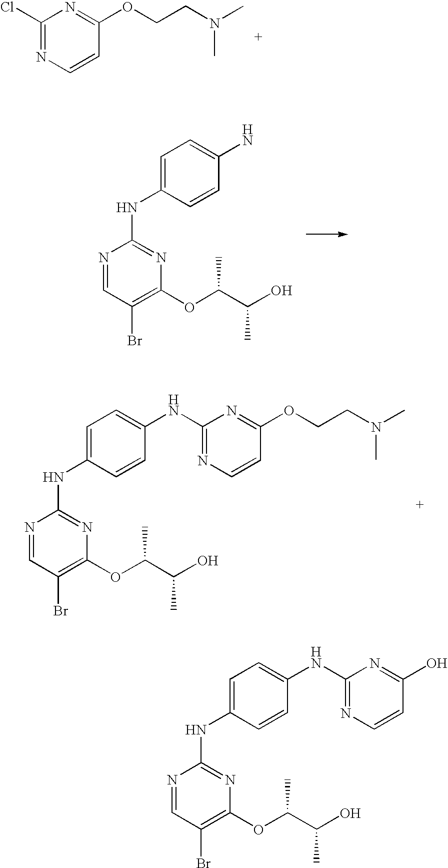

1.5 equivalents of 4 molar hydrochloric acid in dioxane are added to a solution of −[2-(2-chloropyrimidin)-4-yloxy)ethyl]dimethylamine (100 mg, 0.5 mmol) and 175.2 mg (0.5 mmol) of (2R,3R)-3-[2-(4-aminophenylamino)-5-bromopyrimidin-4-yloxy]butan-2-ol in 8.3 ml of 10:1 n-butanol/MeOH, and the mixture is stirred at +60° C. for 24 hours. After cooling, 3 equivalents (0.21 ml) of triethylamine are added, and the mixture is concentrated and then flash chromatographed on silica gel with DCM/MeOH 9:1. 50 mg of a mixture are obtained in this way and are fractionated by HPLC into 19 mg 7.4% of theory of (2R,3R)-3-(5-bromo-2-{4-[4-(2-dimethylaminoethoxy)pyrimidin-2-ylamino]phenylamino}pyrimidin-4-yloxy)butan-2-ol and 26 mg 11.7% of theory of 2-{4-[5-bromo-4-((1R,2R)-2-hydroxy-1-methyl-propoxy)pyrimidin-2-ylamino]phenylamino}pyrimidin-4-ol.

Compound 10

¹H-NMR (DMSO): 9.52 (s, 1H), 9.45 (s, 1H), 8.27 (s, 1H), 8.19 (d, 1H), 7.55 (m, 4H), 6.23 (d, 1H), 5.16 (m, 1H), 4.87 (s, 1H), 4.74 (s, 1H), 3.76 (m, 3H), 3.12 (s 6H), 1.21 (d, 3H), 1.07 (d, 3H)

MS: 520 (Cl).

Compound 11

¹H-NMR (DMSO): 9.66 (s, 1H), 8.30 (s, 1H), 7.66 (m, 3H), 7.41 (d, 2H), 5.84 (d, 1H), 5.15 (m, 2H), 3.78 (m, 1H), 1.23 (d, 3H), 1.07 (d, 3H)

MS: 447 (ES).

### Process Variant B IV:

Substituted anilinopyrimidines of the formula (VIII) can be reacted with nucleophiles of the formula (IX) to give compounds of the formula (I).

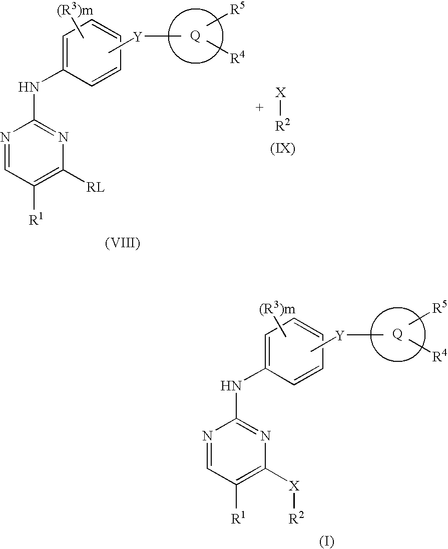

Scheme 11: Preparation of the target compounds by nucleophlic substitution in position 4 of the pyrimidine. The substituents Q, R¹, R², R³, R⁴, R⁵, X, Y and m have the meanings indicated in general formula (I).
   RL is a leaving group such as —F, —Cl, —Br, —I, —OCF₃, —S—CH₃, —SOCH₃ or —SO₂—CH₃.

### EXAMPLES 12-14

### EXAMPLE 12

### (2R,3R)-3-{5-Bromo-2-[4-(pyridin-2-ylamino)phenylamino]pyrimidin-4-ylamino}-butan-2-ol

and

### EXAMPLE 13

### (2R,3R)-3-{5-bromo-2-[4-(1-oxypyridin-2-ylamino)phenylamino]pyrimidin-4-ylamino}butan-2-ol

and

### EXAMPLE 14

### N-(5-bromo-4-methanesulphinylpyrimidin-2-yl)-N′-pyridin-2-ylbenzene-1,4-diamine

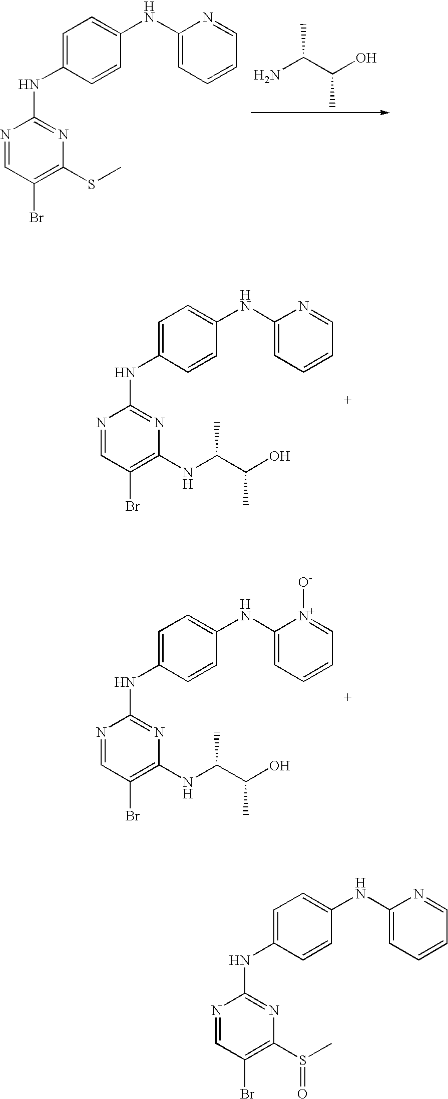

1.5 equivalents (75.3 mg, 0.34 mmol) of m-CPBA are added in portions to a solution of 87 mg (0.22 mmol) of N-(5-bromo-4-methylsulphanylpyrimidin-2-yl)-N′-pyridin-2-yl-benzene-1,4-diamine in 2 ml of N-methyl-2-pyrrolidinone at RT. After stirring at RT for 2 h, 4 equivalents of triethylamine (0.12 ml, 0.9 mmol) and 56 mg (0.45 mmol) of (2R,3R)-3-aminobutan-2-ol are added. After 5 h at +60° C., the reaction mixture is poured into ice-water, extracted 3× with ethyl acetate, dried with sodium sulphate and concentrated. Flash chromatography on silica gel CH2CL2/MeOH 9:1 afforded 14 mg (15% of theory) of compound 12, and 25 mg (25% of theory) of compound 13 and 19 mg (21% of theory) of compound 14.

Compound 12

¹H-NMR (DMSO): 9.75 (m, 1H), 9.60 (s, 1H), 8.05 (s, 1H), 7.96 (d, 1H), 7.75 (m, 1H), 7.63 (d, 2H), 7.42 (d, 2H), 6.59 (d, 1H), 6.82 (m, 1H), 6.51 (m, 1H), 4.03 (m, 1H), 3.73 (m, 1H), 1.14 (d, 3H), 1.03 (d, 3H)

MS: 429 (ES).

Compound 13

¹H-NMR (DMSO): 9.28 (s, 1H), 9.02 (s, 1H), 8.21 (d, 1H), 8.05 (s, 1H), 7.74 (d, 2H), 7.23 (m, 3H), 7.04 (d, 1H), 6.74 (m, 1H), 6.03 (d, 1H), 5.0 (d, 1H), 4.09 (m, 1H), 3.78 (m, 1H), 1.22 (d, 3H), 1.10 (d, 3H)

MS: 445 (ES).

Compound 14

¹H-NMR (DMSO): 9.82 (s, 1H), 9.09 (s, 1H), 8.30 (s, 1H), 8.20 (d, 1H), 7.75 (m, 2H), 7.26 (m, 3H), 7.09 (d, 1H), 6.76 (m, 1H), 2.57 (s, 3H)

MS: 406 (ES).

The following compounds are prepared in analogy to the abovementioned examples:

| Example | Structure | Status/remarks | Preparation |
|---|---|---|---|
| 15 | 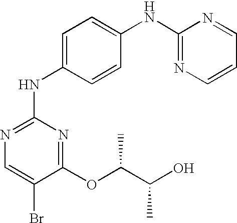 | MS: 430 (ES) | Variant B III |
| 16 | 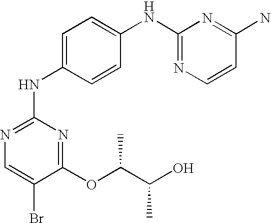 | MS: 446 (ES) | Variant B III |
| 17 | 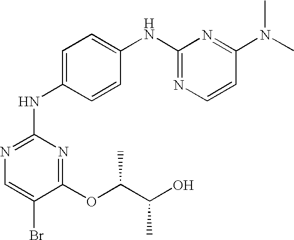 | MS: 474 (ES) | Variant B III |
| 18 | 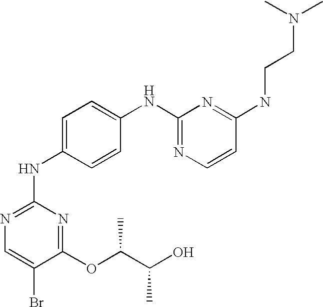 | MS: 519 (ES) | Variant B III |
| 19 | 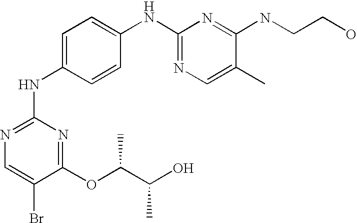 | MS: 504 (ES) | Variant B III |
| 20 | 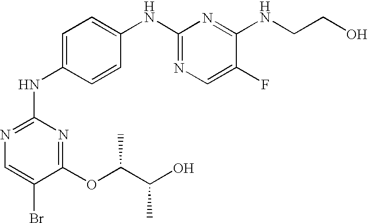 | MS: 508 (ES) | Variant B III |
| 21 | 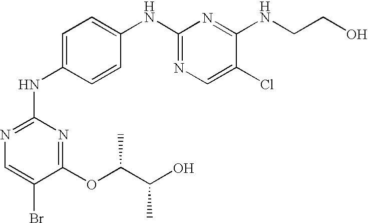 | MS: 524 (ES) | Variant B III |
| 22 | 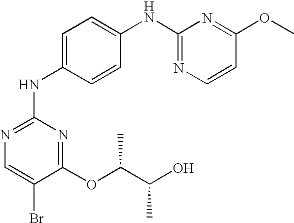 | MS: 461 (ES) | Variant B III |
| 23 | 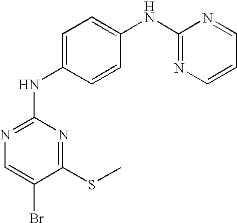 | | IntermediateVIII.1 |
| 24 | 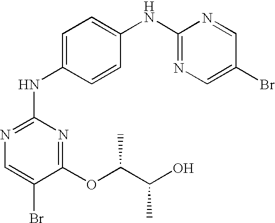 | MS: 509/511/513(ES) | Variant B III |
| 25 | 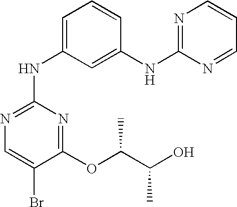 | MS: 431 (ES) | Variant B III |
| 26 | 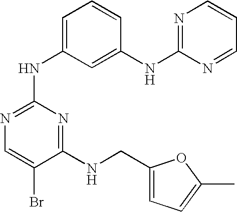 | MS: 452 (ES+) | Variant B III |
| 27 | 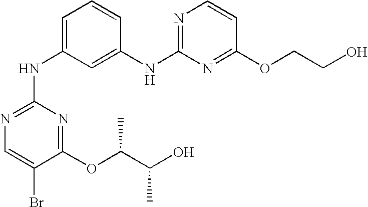 | MS: 491 (ES) | Variant B III |
| 28 | 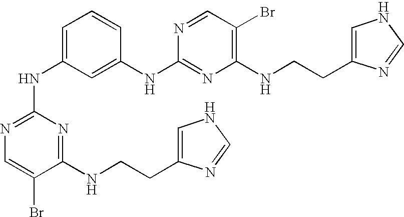 | | Variant B I |
| 29 | 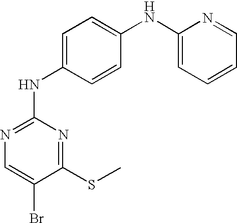 | | IntermediateVIII.2 |
| 30 | 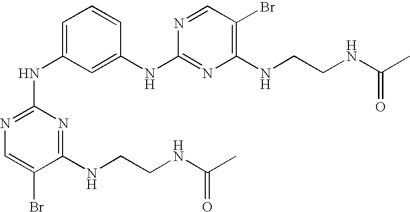 | | Variant B I |
| 31 | 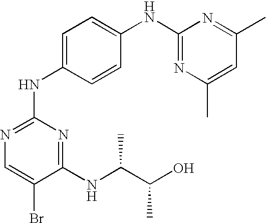 | MS: 458 (ES+) | Variant B II |
| 32 | 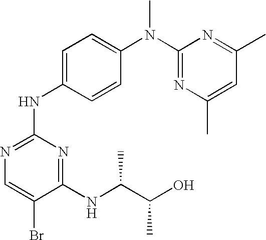 | MS: 472 (ES+) | Variant B II |
| 33 | 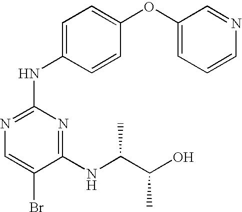 | MS: 430 (ES+) | Variant B II |
| 34 | 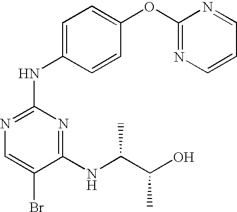 | MS: 431 (ES+) | Variant B II |
| 35 | 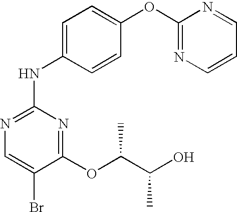 | MS: 432 (ES+) | Variant B II |
| 36 | 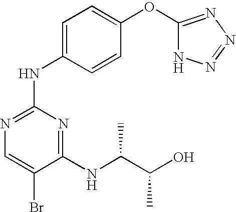 | MS: 421 (ES+) | Variant B II |
| 37 | 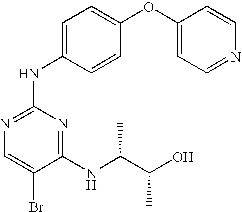 | MS: 430 (ES+) | Variant B II |
| 38 | 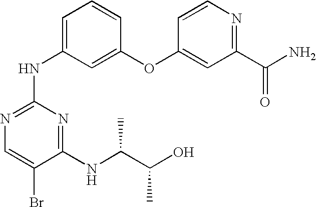 | MS: 473 (ES+) | Variant B II |
| 39 | 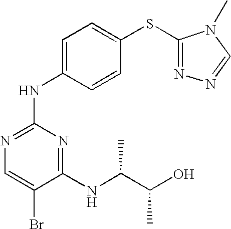 | MS: 450 (ES+) | Variant B II |
| 40 | 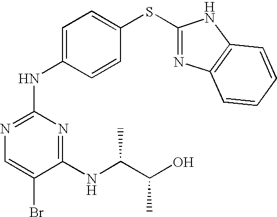 | MS: 485 (ES+) | Variant B II |
| 41 | 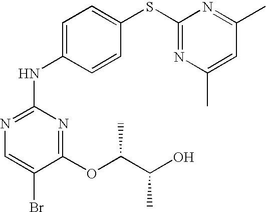 | MS: 475 (ES+) | Variant B II |
| 42 | 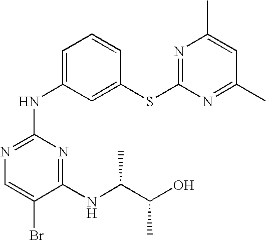 | MS: 475 (ES+) | Variant B II |
| 43 | 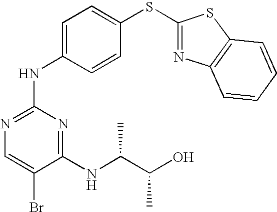 | MS: 502 (ES+) | Variant B II |

The following compounds can be prepared in analogy to the above-mentioned examples:

| | Structure |
|---|---|
| 44 | 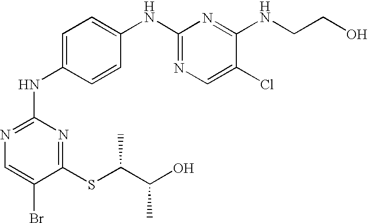 |
| 45 | 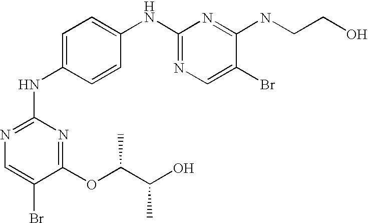 |
| 46 | 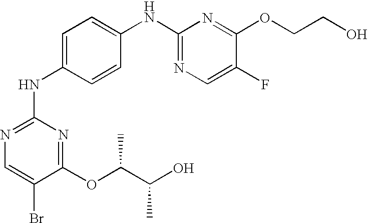 |
| 47 | 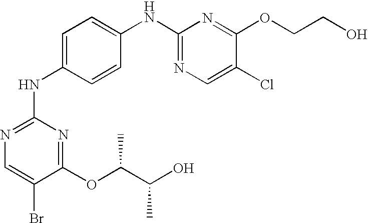 |
| 48 | 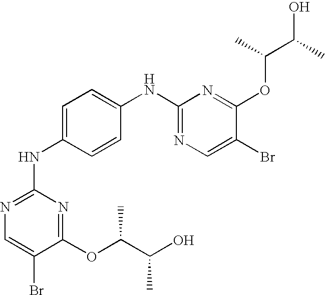 |
| 49 | 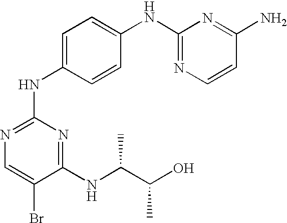 |
| 50 | 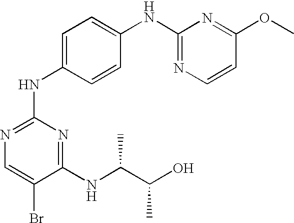 |
| 51 | 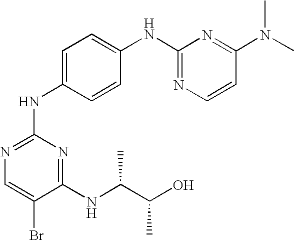 |
| 52 | 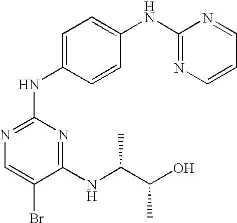 |
| 53 | 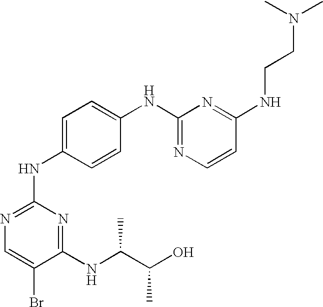 |
| 54 | 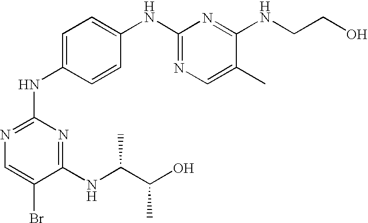 |
| 55 | 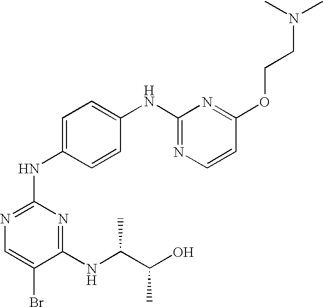 |
| 56 | 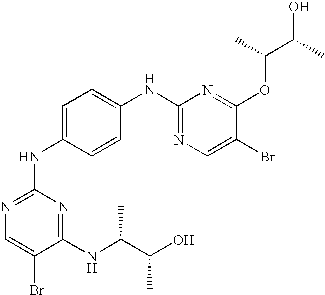 |
| 57 | 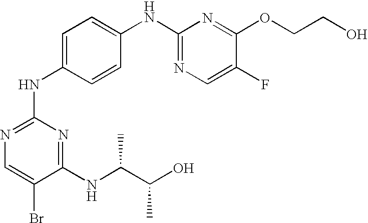 |
| 58 | 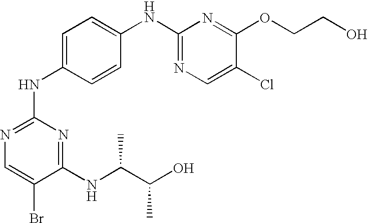 |
| 59 | 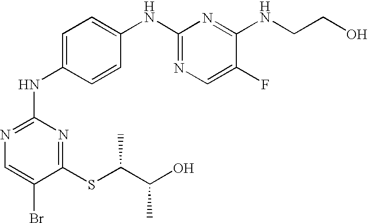 |
| 60 | 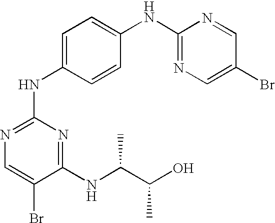 |
| 61 | 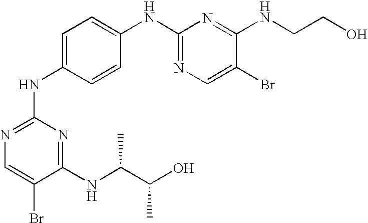 |
| 62 | 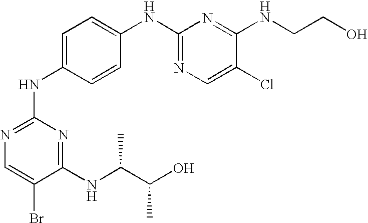 |
| 63 | 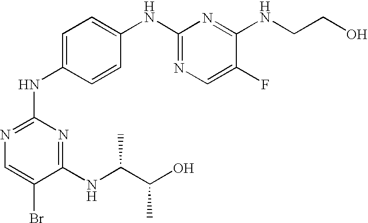 |
| 64 | 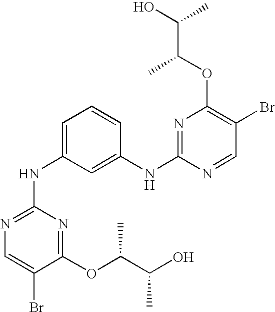 |
| 65 | 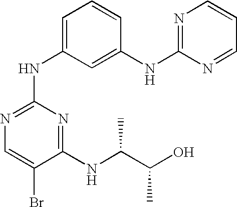 |
| 66 | 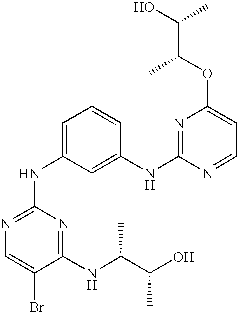 |
| 67 | 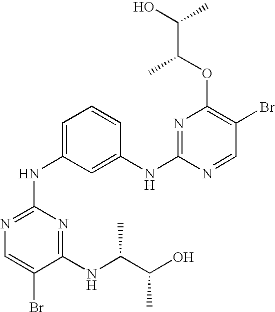 |
| 68 | 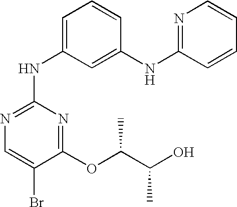 |
| 69 | 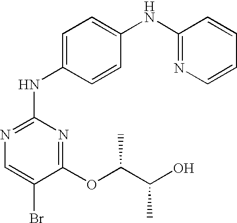 |
| 70 | 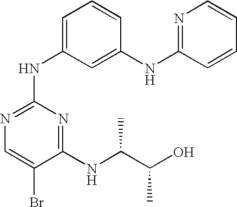 |
| 71 | 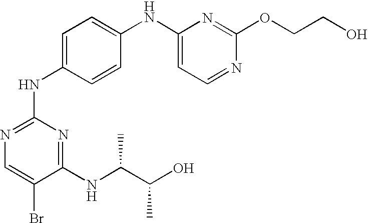 |
| 72 | 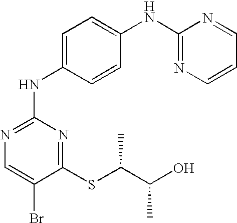 |
| 73 | 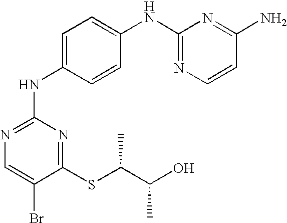 |
| 74 | 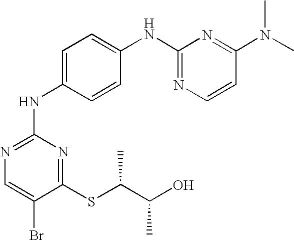 |
| 75 | 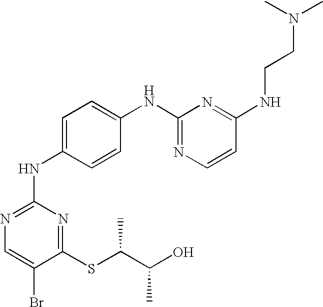 |
| 76 | 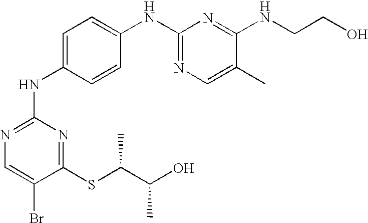 |

The following grouping of protein kinases underlies the application:

A. cell cycle kinases: a) CDKS, b) Plk, c) Aurora
B. angiogenic receptor tyrosine kinases: a) VEGF-R, b) Tie, c) FGF-R, d) EphB4
C. proliferative receptor tyrosine kinases: a) PDGF-R, Flt-3, c-Kit
D. checkpoint kinases: a) AMT/ATR, b) Chk ½, c) TTK/hMps1, BubR1, Bub1
E. anti-apoptotic kinases a) AKT/PKB b) IKK c) PIM1, d) ILK
F. migratory kinases a) FAK, b) ROCK

### A. Cell Cycle Kinases a) CDKs, b) Plk, c) Aurora

The eukaryotic cycle of cell division ensures duplication of the genome and its distribution to the daughter cells by passing through a coordinated and regulated sequence of events. The cell cycle is divided into four consecutive phases: the G1 phase represents the time before DNA replication in which the cell grows and is sensitive to external stimuli. In the S phase, the cell replicates its DNA, and in the G2 phase it prepares itself for entry into mitosis. In mitosis (M phase), the replicated DNA is separated and cell division is completed.

The cyclin-dependent kinases (CDKs), a family of serine/threonine kinases whose members require the binding of a cyclin (Cyc) as regulatory subunit for their activation, drive the cell through the cell cycle. Different CDK/Cyc pairs are active in the different phases of the cell cycle. CDK/Cyc pairs which are important for the basic function of the cell cycle are, for example, CDK4(6)/CycD, CDK2/CycE, CDK2/CycA, CDK1/CycA and CDK1/CycB.

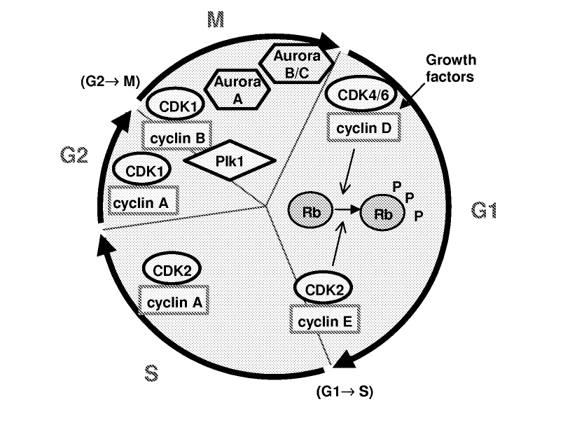

Entry into the cell cycle and passing through the restriction point, which marks the independence of a cell from further growth signals for completion of the initiated cell division, are controlled by the activity of the CDK4(6)/CycD and CDK2/CycE complexes. The essential substrate of these CDK complexes is the retinoblastoma protein (Rb), the product of the retinoblastoma tumour suppressor gene. Rb is a transcriptional corepressor protein. Besides other mechanisms which are still substantially not understood, Rb binds and inactivates transcription factors of the E2F type, and forms transcriptional repressor complexes with histone deacetylases (HDAC) (Zhang H. S. et al. (2000). Exit from G1 and S phase of the cell cycle is regulated by repressor complexes containing HDAC-Rb-hSWI/SNF and Rb-hSWI/SNF. Cell 101, 79-89). Phosphorylation of Rb by CDKs releases bound E2F transcription factors which lead to transcriptional activation of genes whose products are required for DNA synthesis and progression through the S phase. An additional effect of Rb phosphorylation is to break up Rb-HDAC complexes, thus activating further genes. Phosphorylation of Rb by CDKs is to be equated with going beyond the restriction point. The activity of CDK2/CycE and CDK2/CycA complexes is necessary for progression through the S phase and completion thereof. After replication of the DNA is complete, the CDK1 in the complex with CycA or CycB controls the passing through of the G2 phase and the entry of the cell into mitosis (FIG. 1). In the transition from the G2 phase into mitosis, the polo-like kinase Plk1 contributes to activating CDK1. While mitosis is in progress, Plk1 is further involved in the maturation of the centrosomes, the construction of the spindle apparatus, the separation of the chromosomes and the separation of the daughter cells.

The family of Aurora kinases consists in the human body of three members:

Aurora-A, Aurora-B and Aurora-C. The Aurora kinases regulate important processes during cell division (mitosis).

Aurora-A is localized on the centrosomes and the spindle microtubules, where it phosphorylates various substrate proteins, inter alia kinesin Eg5, TACC, PP1. The exact mechanisms of the generation of the spindle apparatus and the role of Aurora-A therein are, however, still substantially unclear.

Aurora-B is part of a multiprotein complex which is localized on the centrosome structure of the chromosomes and, besides Aurora-B, comprises inter alia INCENP, survivin and borealin/dasra B (summarizing overview in: Vagnarelli & Earnshaw, Chromosomal passengers: the four-dimensional regulation of mitotic events. Chromosoma. 2004 November; 113(5): 211-22. Epub 2004 Sep. 4). The kinase activity of Aurora-B ensures that all the connections to the microtubulin spindle apparatus are correct before division of the pairs of chromosomes (so-called spindle checkpoint). Substrates of Aurora-B are in this case inter alia histone H3 and MCAK. After separation of the chromosomes, Aurora-B alters its localization and can be found during the last phase of mitosis (cytokinesis) on the still remaining connecting bridge between the two daughter cells. Aurora-B regulates the severance of the daughter cells through phosphorylation of its substrates MgcRacGAP, vimentin, desmin, the light regulatory chain of myosin, and others.

Aurora-C is very similar in its amino acid sequence, localization, substrate specificity and function to Aurora-B (Li X et al. Direct association with inner centromere protein (INCENP) activates the novel chromosomal passenger protein, Aurora-C. J Biol. Chem. 2004 Nov. 5; 279(45): 47201-11. Epub 2004 Aug. 16; Chen et al. Overexpression of an Aurora-C kinase-deficient mutant disrupts the Aurora-B/INCENP complex and induces polyploidy. J Biomed Sci. 2005; 12(2): 297-310; Yan X et al. Aurora-C is directly associated with Survivin and required for cytokinesis. Genes to ells 2005 10, 617-626). The chief difference between Aurora-B and Aurora-C is the strong overexpression of Aurora-C in the testis (Tseng T C et al. Protein kinase profile of sperm and eggs: cloning and characterization of two novel testis-specific protein kinases (AIE1, AIE2) related to yeast and fly chromosome segregation regulators. DNA Cell Biol. 1998 October; 17(10):823-33.).

The essential function of the Aurora kinases in mitosis makes them target proteins of interest for the development of small inhibitory molecules for the treatment of cancer or other disorders which are caused by disturbances of cell proliferation. Convincing experimental data indicate that inhibition of the Aurora kinases in vitro and in vivo prevents the advance of cellular proliferation and induces programmed cell death (apoptosis). It has been possible to show this by means of (1) siRNA technology (Du & Hannon. Suppression of p160ROCK bypasses cell cycle arrest after Aurora-A/STK15 depletion. Proc Natl Acad Sci USA. 2004 Jun. 15; 101 (24): 8975-80. Epub 2004 Jun. 3; Sasai K et al. Aurora-C kinase is a novel chromosomal passenger protein that can complement Aurora-B kinase function in mitotic cells. Cell Motil Cytoskeleton. 2004 December; 59(4):249-63) or (2) overexpression of a dominant-negative Aurora kinase (Honda et al. Exploring the functional interactions between Aurora B, INCENP, and survivin in mitosis. Mol Biol Cell. 2003 August; 14(8):3325-41. Epub 2003 May 29), and (3) with small chemical molecules which specifically inhibit Aurora kinases (Hauf S et al. The small molecule Hesperadin reveals a role for Aurora B in correcting kinetochore-microtubule attachment and in maintaining the spindle assembly checkpoint. J. Cell Biol. 2003 Apr. 28; 161 (2): 281-94. Epub 2003 Apr. 21; Ditchfield C et al. Aurora B couples chromosome alignment with anaphase by targeting BubR1, Mad2, and Cenp-E to kinetochores. J. Cell Biol. 2003 Apr. 28; 161 (2): 267-80.).

Inactivation of Aurora kinases leads to (1) faulty or no development of the mitotic spindle apparatus (predominantly with Aurora-A inhibition) and/or (2) faulty or no separation of the sister chromatids through blocking of the spindle checkpoint (predominantly with Aurora-B/-C inhibition) and/or (3) incomplete separation of daughter cells (predominantly with Aurora-B/-C inhibition). These consequences (1-3) of the inactivation of Aurora kinases singly or as combinations lead eventually to aneuploidy and/or polyploidy and ultimately, immediately or after repeated mitoses, to a non-viable state or to programmed cell death of the proliferating cells (mitotic catastrophe).

Specific kinase inhibitors are able to influence the cell cycle at various stages. Thus, for example, blockade of the cell cycle in the G1 phase or in the transition from the G1 phase to the S phase is to be expected with a CDK4 or a CDK2 inhibitor.

### B. Angiogenic Receptor Tyrosine Kinases

Receptor tyrosine kinases and their ligands are crucial participants in a large number of cellular processes involved in the regulation of the growth and differentiation of cells. Of particular interest here are the vascular endothelial growth factor (VEGF)/VEGF receptor system, the fibroblast growth factor (FGF)/FGF receptor system, the Eph ligand/Eph receptor system, and the Tie ligand/Tie receptor system. In pathological situations associated with an increased formation of new blood vessels (neovascularization) such as, for example, neoplastic diseases, an increased expression of angiogenic growth factors and their receptors has been found. Inhibitors of the VEGF/VEGF receptor system, FGF/FGF receptor system (Rousseau et al., The tyrp1-Tag/tyrp1-FGFR1-DN bigenic mouse: a model for selective inhibition of tumor development, angiogenesis, and invasion into the neural tissue by blockade of fibroblast growth factor receptor activity. Cancer Res. 64: 2490, 2004), of the EphB4 system (Kertesz et al., The soluble extracellular domain of EphB4 (sEphB4) antagonizes EphB4-EphrinB2 interaction, modulates angiogenesis and inhibits tumor growth. Blood. 2005 Dec. 1; [Epub ahead of print]), and of the Tie ligand/Tie system (Siemeister et al., Two independent mechanisms essential for tumor angiogenesis: inhibition of human melanoma xenograft growth by interfering with either the vascular endothelial growth factor receptor pathway or the Tie-2 pathway. Cancer Res. 59, 3185, 1999) are able to inhibit the development of a vascular system in tumours, thus cut the tumour off from the oxygen and nutrient supply, and therefore inhibit tumour growth.

### C. Proliferative Receptor Tyrosine Kinases

Receptor tyrosine kinases and their ligands are crucial participants in the proliferation of cells. Of particular interest here are the platelet-derived growth factor (PDGF) ligand/PDGF receptor system, c-kit ligand/c-kit receptor system and the FMS-like tyrosine kinase 3 (Flt-3) ligand/Flt-3 system. In pathological situations associated with an increased growth of cells such as, for example, neoplastic diseases, an increased expression of proliferative growth factors and their receptors or kinase-activating mutations has been found. Inhibition of the enzymic activity of these receptor tyrosine kinases leads to a reduction of tumour growth. It has been possible to show this for example by studies with the small chemical molecule STI571/Glivec which inhibits inter alia PDGF-R and c-kit (summarizing overviews in: Oestmann A., PDGF receptors—mediators of autocrine tumor growth and regulators of tumor vasculature and stroma, Cytokine Growth Factor Rev. 2004 August; 15(4):275-86; Roskoski R., Signaling by Kit protein-tyrosine kinase—the stem cell factor receptor. Biochem Biophys Res Commun. 2005 Nov. 11; 337(1): 1-13; Markovic A. et al., FLT-3: a new focus in the understanding of acute leukemia. Int J Biochem Cell Biol. 2005 June; 37(6):1168-72. Epub 2005 Jan. 26.).

### E. Checkpoint Kinases

Checkpoint kinases mean in the context of the present application cell cycle kinases which monitor the ordered progression of cell division, such as, for example, ATM and ATR, Chk1 and Chk2, Mps1, Bub1 and BubR1. Of particular importance are the DNA damage checkpoint in the G2 phase and the spindle checkpoint during mitosis.

The ATM, ATR, Chk1 and Chk2 kinases are activated by DNA damage to a cell and leads to arrest of the cell cycle in the G2 phase through inactivation of CDK1. (Chen & Sanchez, Chk1 in the DNA damage response: conserved roles from yeasts to mammals. DNA Repair 3, 1025, 2004). Inactivation of Chk1 causes loss of the G2 arrest induced by DNA damage, to progression of the cell cycle in the presence of damaged DNA, and finally leads to cell death (Takai et al. Aberrant cell cycle checkpoint function and early embryonic death in Chk1 (−/−) mice. Genes Dev. 2000 Jun. 15; 14(12): 1439-47; Koniaras et al. Inhibition of Chk1-dependent G2 DNA damage checkpoint radiosensitizes p53 mutant human cells. Oncogene. 2001 Nov. 8; 20(51): 7453-63; Liu et al. Chk1 is an essential kinase that is regulated by Atr and required for the G(2)/M DNA damage checkpoint. Genes Dev. 2000 Jun. 15; 14(12): 1448-59.). Inactivation of Chk1, Chk2 or Chk1 and Chk2 prevents the G2 arrest caused by DNA damage and makes proliferating cancer cells more sensitive to DNA-damaging therapies such as, for example, chemotherapy or radiotherapy. Chemotherapies leading to DNA damage are, for example, substances inducing DNA strand breaks, DNA-alkylating substances, topoisomerase inhibitors, Aurora kinase inhibitors, substances which influence the construction of the mitotic spindles, hypoxic stress owing to a limited oxygen supply to a tumour (e.g. induced by anti-angiogenic medicaments such as VEGF kinase inhibitors).

A second essential checkpoint within the cell cycle controls the correct construction and attachment of the spindle apparatus to the chromosomes during mitosis. The kinases TTK/hMps1, Bub1, and BubR1 are involved in this so-called spindle checkpoint (summarizing overview in: Kops et al. On the road to cancer: aneuploidy and the mitotic checkpoint. Nat Rev Cancer. 2005 October; 5(10):773-85). These are localized on kinetochores of condensed chromosomes which are not yet attached to the spindle apparatus and inhibit the so-called anaphase-promoting complex/cyclosome (APC/C). Only after complete and correct attachment of the spindle apparatus to the kinetochores are the spindle checkpoint kinases Mps-1, Bub1, and BubR1 inactivated, thus activating APC/C and resulting in separation of the paired chromosomes. Inhibition of the spindle checkpoint kinases leads to separation of the paired chromosomes before all the kinetochores are attached to the spindle apparatus, and consequently to faulty chromosome distributions which are not tolerated by cells and finally lead to cell cycle arrest or cell death.

### F. Anti-Apoptotic Kinases

Various mechanisms protect a cell from cell death during non-optimal living conditions. In tumour cells, these mechanisms lead to a survival advantage of the cells in the growing mass of the tumour, which is characterized by deficiency of oxygen, glucose and further nutrients, make it possible for tumour cells to survive without attachment to the extracellular matrix, possibly leading to metastasis, or lead to resistances to therapeutic agents. Essential anti-apoptotic signalling pathways include the PDK1-AKT/PKB signalling pathway (Altomare & Testa. Perturbations of the AKT signaling pathway in human cancer. Oncogene. 24, 7455, 2005), the NFkappaB signalling pathway (Viatour et al. Phosphorylation of NFkB and IkB proteins: implications in cancer and inflammation), the Pim1 signalling pathway (Hammerman et al. Pim and Akt oncogenes are independent regulators of hematopoietic cell growth and survival. Blood. 2005 105, 4477, 2005) and the integrin-linked kinase (ILK) signalling pathway (Persad & Dedhar. The role of integrin-linked kinase (ILK) in cancer progression. Cancer Met. Rev. 22, 375, 2003). Inhibition of the anti-apoptotic kinases such as, for example, AKT/PBK, PDK1, IkappaB kinase (IKK), Pim1, or ILK sensitizes the tumour cells to the effect of therapeutic agents or to unfavourable living conditions in the tumour environment. After inhibition of the anti-apoptotic kinases, tumour cells will react more sensitively to disturbances of mitosis caused by Aurora inhibition and undergo cell death in increased numbers.

### G. Migratory Kinases

A precondition for invasive, tissue-infiltrating tumour growth and metastasis is that the tumour cells are able to leave the tissue structure through migration. Various cellular mechanisms are involved in regulating cell migration: integrin-mediated adhesion to proteins of the extracellular matrix regulates via the activity of focal adhesion kinase (FAK); control of the assembling of contractile actin filaments via the RhoA/Rho kinase (ROCK) signalling pathway (summarizing overview in M. C. Frame, Newest findings on the oldest oncogene; how activated src does it. J. Cell Sci. 117, 989, 2004).

The compounds according to the invention are effective for example
against cancer such as solid tumours, tumour growth or metastasis growth, especially:
ataxia-telangiectasia, basal cell carcinoma, bladder carcinoma, brain tumour, breast cancer, cervical carcinoma, tumours of the central nervous system, colorectal carcinoma, endometrial carcinoma, stomach carcinoma, gastrointestinal carcinoma, head and neck tumours, acute lymphocytic leukaemia, acute myelogenous leukaemia, chronic lymphocytic leukaemia, chronic myelogenous leukaemia, hairy cell leukaemia, liver carcinoma, lung tumour, non-small-cell lung carcinoma, small-cell lung carcinoma, B-cell lymphoma, Hodgkin's lymphoma, non-Hodgkin's lymphoma, T-cell lymphoma, melanoma, mesothelioma, myeloma, myoma, tumours of the oesophagus, oral tumours, ovarian carcinoma, pancreatic tumours, prostate tumours, renal carcinoma, sarcoma, Kaposi's sarcoma, leiomyosarcoma, skin cancer, squamous cell carcinoma, testicular cancer, thyroid cancer, connective tissue tumour of the gastrointestinal tissue, connective tissue sarcoma of the skin, hypereosinophilic syndrome, mast cell cancer,
for cardiovascular disorders such as stenoses, arterioscleroses and restenoses, stent-induced restenosis,
for angiofibroma, Crohn's disease, endometriosis, haemangioma.

Formulation of the compounds according to the invention to give pharmaceutical products takes place in a manner known per se by converting the active ingredient(s) with the excipients customary in pharmaceutical technology into the desired administration form.

Excipients which can be employed in this connection are, for example, carrier substances, fillers, disintegrants, binders, humectants, lubricants, absorbents and adsorbents, diluents, solvents, cosolvents, emulsifiers, solubilizers, masking flavours, colorants, preservatives, stabilizers, wetting agents, salts to alter the osmotic pressure or buffers. Reference should be made in this connection to Remington's Pharmaceutical Science, 15th ed. Mack Publishing Company, East Pennsylvania (1980).

The pharmaceutical formulations may be

in solid form, for example as tablets, coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may be, for example, salts, saccharides (mono-, di-, tri-, oligo- and/or polysaccharides), proteins, amino acids, peptides, fats, waxes, oils, hydrocarbons and their derivatives, where the excipients may be of natural origin or may be obtained by synthesis or partial synthesis.

Suitable for oral or peroral administration are in particular tablets, coated tablets, capsules, pills, powders, granules, pastilles, suspensions, emulsions or solutions. Suitable for parenteral administration are in particular suspensions, emulsions and especially solutions.

### Description of the Assays

### Assay 1

### Aurora-C Kinase Assay

The Aurora-C inhibitory activity of the substances of this invention was measured in the Aurora-C-HTRF assay (HTRF=Homogeneous Time Resolved Fluorescence) described in the following paragraphs.

Recombinant fusion protein of GST and human Aurora-C was expressed in transiently transfected HEK293 cells and purified by affinity chromatography on glutathione-Sepharose. The substrate used for the kinase reaction was the biotinylated peptide biotin-Ttds-FMRLRRLSTKYRT (C terminus in amide form) which can be purchased for example from JERINI Peptide Technologies (Berlin). Aurora-C was incubated in the presence of various concentrations of test substances in 5 μL of assay buffer [25 mM Hepes/NaOH pH 7.4, 0.5 mM MnCl₂, 2.0 mM dithiothreitol, 0.1 mM sodium orthovanadate, 10 μM adenosine triphosphate (ATP), 0.5 μM/ml substrate, 0.01% (v/v) TritonX-100 (Sigma), 0.05% (w/v) bovine serum albumin (BSA), 1% (v/v) dimethyl sulphoxide] at 22° C. for 60 min. The concentration of Aurora-C was adapted to the particular activity of the enzyme and adjusted so that the assay operated in the linear range. Typical concentrations were in the region of 0.3 nM. The reaction was stopped by adding 5 μl of a solution of HTRF detection reagents (0.2 μM streptavidin-XLent and 1.4 nM anti-phospho-(Ser/Thr)-Akt substrate-Eu-cryptate (C is biointernational, France, product No. 61P02KAE), a Europium-cryptate-labelled phospho-(Ser/Thr)-Akt substrate antibody [product #9611B, Cell Signaling Technology, Danvers, Mass., USA]) in aqueous EDTA solution (40 mM EDTA, 400 mM KF, 0.05% (w/v) bovine serum albumin (BSA) in 25 mM HEPES/NaOH pH 7.0).

The resulting mixture was incubated at 22° C. for 1 h in order to allow formation of a complex of the biotinylated phosphorylated substrate and the detection reagents. The amount of phosphorylated substrate was then estimated by measuring the resonance energy transfer from the anti-phospho-(Ser/Thr)-Akt substrate-Eu cryptate to the streptavidin-XLent. For this purpose, the fluorescence emissions at 620 nm and 665 nm after excitation at 350 nm were measured in an HTRF measuring instrument, e.g. a Rubystar (BMG Labtechnologies, Offenburg, Germany) or a Viewlux (Perkin-Elmer). The ratio of the emissions at 665 nm and at 622 nm was taken as a measure of the amount of phosphorylated substrate. The data were normalized (enzymic reaction without inhibitor=0% inhibition, all other assay components but no enzyme=100% inhibition) and IC50 values were calculated with a 4-parameter fit using an inhouse software.

### Assay 2

### CDK1/CycB Kinase Assay

Recombinant CDK1- and CycB-GST fusion proteins, purified from baculovirus-infected insect cells (Sf9), were purchased from ProQinase GmbH, Freiburg. The histone IIIS used as kinase substrate can be purchased from Sigma.

CDK1/CycB (5 ng/μL) was incubated in the presence of various concentrations of test substances (0 μM, and within the range 0.01-100 μM) in 40 μL of assay buffer [50 mM Tris/HCl pH 8.0, 10 mM MgCl₂, 0.1 mM Na ortho-vanadate, 1.0 mM dithiothreitol, 0.025% PEG 20000, 0.5 μM ATP, 10 μM histone IIIIS, 0.2 μCi/measurement point ³³P-gamma ATP, 0.05% NP40, 1.25% dimethyl sulphoxide] at 22° C. for 10 min. The reaction was stopped by adding EDTA solution (250 mM, pH 8.0, 15 μl/measurement point).

15 μl of each reaction mixture were loaded onto P30 filter strips (from Wallac), and non-incorporated ³³P-ATP was removed by washing the filter strips three times in 0.5% strength phosphoric acid for 10 min each time. After the filter strips had been dried at 70° C. for 1 hour, the filter strips were covered with scintillator strips (MeltiLex™ A, from Wallac) and baked at 90° C. for 1 hour. The amount of incorporated ³³P (substrate phosphorylation) was determined by scintillation measurement in a gamma radiation counter (Wallac).

The measured data were normalized to 0% inhibition (enzyme reaction without inhibitor) and 100% inhibition (all assay components except enzyme). The IC50 values were determined by means of a 4-parameter fit using the company's own software.

### Assay 3

### CDK2/CycE Kinase Assay

The CDK2/CycE inhibitory activity of the substances of this invention was measured in the CDK2/CycE-HTRF assay (HTRF=Homogeneous Time Resolved Fluorescence) described in the following paragraphs.

Recombinant CDK2-GST and CycE-GST fusion proteins purified from baculovirus-infected insect cells (Sf9) were purchased from ProQinase GmbH, Freiburg. The substrate used for the kinase reaction was the biotinylated peptide biotin-Ttds-YISPLKSPYKISEG (C terminus in amide form) which can be purchased for example from JERINI Peptide Technologies (Berlin).

CDK2/CycE was incubated in the presence of various concentrations of test substances in 5 μL of assay buffer [50 mM Tris/HCl pH 8.0, 10 mM MgCl₂, 1.0 mM dithiothreitol, 0.1 mM sodium orthovanadate, 10 μM adenosine triphosphate (ATP), 0.75 μM substrate, 0.01% (v/v) Nonidet-P40 (Sigma), 1% (v/v) dimethyl sulphoxide] at 22° C. for 60 min. The concentration of CDK2/CycE was adapted to the particular activity of the enzyme and adjusted so that the assay operated in the linear range. Typical concentrations were in the region of 1 ng/ml. The reaction was stopped by adding 5 μl of a solution of HTRF detection reagents (0.2 μM streptavidin-XLent and 3.4 nM phospho-(Ser) CDKs substrate antibody [product #2324B, Cell Signaling Technology, Danvers, Mass., USA] and 4 nM Prot-A-EuK [protein A labelled with Europium cryptate from C is biointernational, France, product No. 61 PRAKLB]) in aqueous EDTA solution (100 mM EDTA, 800 mM KF, 0.2% (w/v) bovine serum albumin (BSA) in 100 mM HEPES/NaOH pH 7.0).

The resulting mixture was incubated at 22° C. for 1 h in order to allow formation of a complex of the biotinylated phosphorylated substrate and the detection reagents. The amount of phosphorylated substrate was then estimated by measuring the resonance energy transfer from the Prot-A-EuK to the streptavidin-XLent. For this purpose, the fluorescence emissions at 620 nm and 665 nm after excitation at 350 nm were measured in an HTRF measuring instrument, e.g. a Rubystar (BMG Labtechnologies, Offenburg, Germany) or a Viewlux (Perkin-Elmer). The ratio of the emissions at 665 nm and at 622 nm was taken as a measure of the amount of phosphorylated substrate. The data were normalized (enzymic reaction without inhibitor=0% inhibition, all other assay components but no enzyme=100% inhibition) and IC50 values were calculated with a 4-parameter fit using an inhouse software.

### Assay 4

### KDR Kinase Assay

The KDR-inhibitory activity of the substances of this invention was measured in the KDR-HTRF assay (HTRF=Homogeneous Time Resolved Fluorescence) described in the following paragraphs.

Recombinant KDR kinase-GST fusion protein purified from baculovirus-infected insect cells (Sf9) was purchased from ProQinase GmbH, Freiburg. The substrate used for the kinase reaction was the biotinylated peptide biotin-Ahx-DFGLARDMYDKEYYSVG (C terminus in acid form) which can be purchased for example from Biosynthan GmbH (Berlin-Buch, Germany).

KDR kinase was incubated in the presence of various concentrations of test substances in 5 μL of assay buffer [50 mM Hepes/NaOH pH 7.0, 25 mM MgCl₂, 5 mM MnCl₂, 1.0 mM dithiothreitol, 0.1 mM sodium orthovanadate, 10 μM adenosine triphosphate (ATP), 0.5 μM substrate, 0.001% (v/v) Nonidet-P40 (Sigma), 1% (v/v) dimethyl sulphoxide] at 22° C. for 45 min. The concentration of KDR was adapted to the particular activity of the enzyme and adjusted so that the assay operated in the linear range. The reaction was stopped by adding 5 μl of a solution of HTRF detection reagents (0.1 μM streptavidin-XLent and 2 nM PT66-Eu chelate, a Europium chelate-labelled anti-phospho-tyrosine antibody from Perkin Elmer) in aqueous EDTA solution (125 mM EDTA, 0.2% (w/v) bovine serum albumin (BSA) in 50 mM HEPES/NaOH pH 7.0).

The resulting mixture was incubated at 22° C. for 1 h in order to allow formation of the biotinylated phosphorylated substrate and the streptavidin-XLent and PT66-Eu chelate. The amount of phosphorylated substrate was then estimated by measuring the resonance energy transfer from the PT66-Eu chelate to the streptavidin-XLent. For this purpose, the fluorescence emissions at 620 nm and 665 nm after excitation at 350 nm were measured in an HTRF measuring instrument, e.g. a Rubystar (BMG Labtechnologies, Offenburg, Germany) or a Viewlux (Perkin-Elmer). The ratio of the emissions at 665 nm and at 622 nm was taken as a measure of the amount of phosphorylated substrate. The data were normalized (enzymic reaction without inhibitor=0% inhibition, all other assay components but no enzyme=100% inhibition) and IC50 values were calculated with a 4-parameter fit using an inhouse software.

### Assay 5

### MCF7 Proliferation Assay

Cultivated human MCF7 breast tumour cells (ATCC HTB-22) were plated out in a density of 5000 cells/measurement point in 200 μl of growth medium (RPMI1640, 10% foetal calf serum, 2 mU/mL insulin, 0.1 nM oestradiol) in a 96-well multititre plate. After 24 hours, the cells from a plate (zero plate) were stained with crystal violet (see below), while the medium in the other plates was replaced by fresh culture medium (200 μl) to which the test substances had been added in various concentrations (0 μM, and in the range 0.01-30 μM; the final concentration of the solvent dimethyl sulphoxide was 0.5%). The cells were incubated in the presence of the test substances for 4 days. The cell proliferation was determined by staining the cells with crystal violet: the cells were fixed by adding 20 μl/measurement point of an 11% strength glutaraldehyde solution at room temperature for 15 min. After the fixed cells had been washed three times with water, the plates were dried at room temperature. The cells were stained by adding 100 μl/measurement point of a 0.1% strength crystal violet solution (pH adjusted to pH 3 by adding acetic acid). After the stained cells had been washed three times with water, the plates were dried at room temperature. The dye was dissolved by adding 100 μl/measurement point of a 10% strength acetic acid solution, and the extinction was determined by photometry at a wavelength of 595 nm. The percentage change in cell growth was calculated by normalizing the measurements to the extinctions of the zero point plate (=0%) and the extinction of the untreated (0 μM) cells (=100%). The IC50 values were determined by means of a 4-parameter fit using the company's own software.

### Inhibitory Effect of the Compounds According to the Invention

The compounds of the following examples were tested in the various kinase assays for their inhibitory effect and in the MCF7 cancer cell line for their growth-inhibiting effect (Tab. 1)

TABLE 1

| Example | CDK1 | CDK2 | Aurora | KDR | MCF7 |
|---|---|---|---|---|---|
| No | [M] | [M] | C [M] | [M] | [M] |
| 8 | 2.8E−08 | 2.8E−09 | 2E−07 | 4.4E−08 | 3E−08 |
| 15 | 2.7E−08 | 2.9E−09 | 1.4E−07 | 7.4E−08 | 8.9E−08 |
| 16 | 2.3E−08 | 2E−09 | 4.2E−08 | 8.3E−08 | 4.2E−07 |
| 17 | 7.1E−08 | 4.4E−09 | 1E−07 | 6.2E−08 | 9.8E−08 |
| 18 | 4E−08 | 5.9E−09 | 7.5E−08 | 1.4E−07 | 1.7E−07 |
| 19 | 5.1E−08 | 2.2E−09 | 9.1E−08 | 1E−07 | 2.9E−07 |
| 21 | 5.4E−08 | 2.2E−09 | 1.8E−07 | 7.3E−08 | 9.1E−08 |
| 22 | 1.3E−07 | 4.8E−09 | 2.7E−07 | 1.2E−07 | 1.3E−07 |
| 24 | 1.1E−07 | 6.2E−09 | 1.3E−07 | 2.4E−07 | 1E−07 |
| 25 | 1.4E−08 | 2E−09 | 3.4E−07 | 7.5E−08 | 2.7E−07 |
| 27 | 1.6E−08 | 2.4E−09 | 4.4E−07 | 3.2E−08 | 3.3E−08 |
| 28 | | 6.7E−07 | 1.2E−07 | 2.3E−08 | |
| 29 | >1E−06 | 2.6E−07 | 8.1E−06 | >0.00002 | >3E−06 |
| 30 | | 5.8E−06 | 5.3E−08 | 2.1E−08 | 2E−06 |
| 33 | 7.8E−08 | 6.6E−09 | 3.8E−07 | 5E−08 | 2.7E−07 |
| 34 | 3.5E−08 | 9.1E−09 | 4.9E−07 | 1E−07 | |
| 39 | 1.1E−08 | 1.3E−09 | 3.5E−07 | 4.1E−08 | |
| 41 | 9.8E−08 | 6E−08 | | 1.4E−06 | 8E−07 |
| 44 | >1E−06 | 3.7E−07 | 1E−06 | 7.3E−06 | >3E−06 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 06077236.5, filed Dec. 20, 2006, and U.S. Provisional Application Ser. No. 60/880,031, filed Jan. 12, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula (I) with building blocks A, B, C and D, 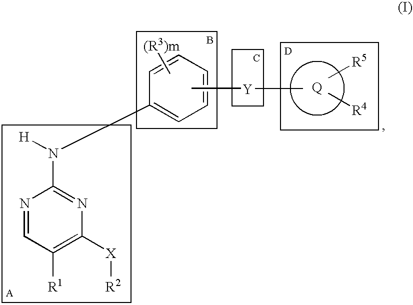
in which
R¹ is halogen, —CF₃, —OCF₃, C₁-C₄-alkyl or nitro,
R² is a C₁-C₁₀-alkyl, C₂-C₁₀-alkenyl or C₂-C₁₀-alkynyl radical, a C₃-C₇-cycloalkyl or a heterocyclyl ring having 3 to 7 ring atoms, in each case optionally substituted one or more times, identically or differently, by hydroxy, —NR⁸R⁹, —NR⁷—C(O)—R¹², —NR⁷—C(O)—OR¹², —NR⁷—C(O)—NR⁸R⁹ or a monocyclic or bicyclic heteroaryl which is optionally itself substituted one or more times by hydroxy or a C₁-C₆-alkyl radical.
R³ is
(i) hydroxy, halogen, cyano, nitro, —CF₃, —OCF₃, —C(O)NR⁸R⁹, —C(S)NR⁸R⁹, —NR⁸R⁹, —NR⁷—C(O)—R², —NR⁷—C(O)—OR², —NR⁷—C(O)—NR⁸R⁹ or —NR⁷—SO₂—R¹² and/or
(ii) a C₁-C₅-alkyl and/or C₁-C₅-alkoxy radical which is optionally substituted one or more times, identically or differently, by halogen, hydroxy, C₁-C₆-alkoxy, —CF₃, —OCF₃ or —NR⁸R⁹,
m is 0-3,
R⁴ and R⁵ are independently of one another
(iii) hydrogen, —NHR⁸, —OR⁸, halogen, —(CO)—NR⁸R⁹ and/or
(iv) a C₁-C₄-alkyl, C₃-C₅-alkenyl, C₃-C₅-alkynyl radical or a C₃-C₆-cycloalkyl ring, in each case optionally themselves substituted one or more times, identically or differently, by hydroxy, —NR⁸R⁹, cyano, halogen, —CF₃, C₁-C₆-alkoxy, —OCF₃ and/or C₁-C₆-alkyl,
X and Y are independently of one another
—O—, —S—, —S(O)—, —S(O)₂— or —NR⁵—, where
R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃, or
if X is —NR¹⁵—,
—NR¹⁵— and R² preferably alternatively together form a 3 to 6 membered ring which, in addition to the nitrogen atom, optionally comprises a further heteroatom, is optionally substituted one or more times, identically or differently, by hydroxy, C₁-C₆-alkyl, C₁-C₆-alkoxy,
—C(O)R¹², —SO₂R¹², halogen or the group —NR⁸R⁹, optionally comprises 1 or 2 double bonds, and/or is interrupted by a —C(O) group.
Q is a monocyclic or bicyclic heteroaryl ring,
R⁶ is
(iii) hydrogen or
(iv) a C₁-C₄-alkyl, C₃-C₅-alkenyl, C₃-C₅-alkynyl or C₁-C₅-alkoxy radical, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃.
R⁷ is hydrogen,
R⁸ and R⁹ are independently of one another
hydrogen and/or a C₁-C₅-alkyl, C₂-C₅-alkenyl radical, a C₃-C₇-cycloalkyl and/or phenyl ring and/or a monocyclic heteroaryl ring, in each case optionally substituted one or more times, identically or differently, by hydroxy, —NR¹⁰R¹¹, —NR⁷—C(O)—R¹² and/or C₁-C₆-alkoxy, or
R⁸ and R⁹ form together with the nitrogen atom a 5- to 7-membered ring which, in addition to the nitrogen atom, optionally comprises 1 further heteroatom and which may be substituted one or more times, identically or differently, by hydroxy, —NR¹⁰R¹¹ and/or C₁-C₆-alkoxy.
R¹⁰ and R¹¹ are independently of one another hydrogen or a C₁-C₆-alkyl radical which is optionally substituted one or more times, identically or differently, by hydroxy, halogen or C₁-C₆-alkoxy,
R¹² is a C₁-C₆-alkyl, C₂-C₆-alkenyl or C₂-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy.
R¹³ and R¹⁴ are independently of one another a C₁-C₆-alkyl, C₂-C₆-alkenyl and/or C₂-C₆-alkynyl radical, a C₃-C₇-cycloalkyl and/or phenyl ring, a heterocyclyl ring having 3 to 8 ring atoms and/or a monocyclic heteroaryl ring, in each case optionally themselves substituted one or more times, identically or differently, by hydroxy, —NR⁸R⁹ and/or C₁-C₆-alkoxy.
R¹⁶ is a C₁-C₆-alkyl, C₃-C₆-alkenyl, C₃-C₆-alkynyl radical, a C₃-C₇-cycloalkyl or phenyl ring, a heterocyclyl ring having 3 to 8 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
and the salts, diastereomers and enantiomers thereof.

2. Compounds of the formula (I) according to claim 1, in which Y is —O—, —S— or
—NR¹⁵—, where
R¹⁵ is hydrogen or a C₁-C₆-alkyl radical, C₃-C₈-cycloalkyl or a heterocyclyl ring having 3 to 8 ring atoms, in each case optionally substituted one or more times, identically or differently, by hydroxy, —NR¹⁰R¹¹, cyano, halogen, —CF₃, C₁-C₆-alkoxy and/or —OCF₃.

3. Compounds of the formula (I) according to claim 1, in which
R¹ is halogen, —CF₃ or C₁-C₂-alkyl,
and the salts, diastereomers and enantiomers thereof.

4. Compounds of the formula (I) according to claim 1, in which
R² is a C₁-C₈-alkyl, C₂-C₈-alkenyl or C₂-C₈-alkynyl radical, a C₃-C₆-cycloalkyl or a heterocyclyl ring having 3 to 5 ring atoms, in each case optionally substituted one or more times, identically or differently, by hydroxy, —NR⁸R⁹, —NR⁷—C(O)—R¹², —NR⁷—C(O)—OR¹², —NR⁷—C(O)—NR⁸R⁹ or a monocyclic heteroaryl which is optionally itself substituted one or more times by hydroxy or a C₁-C₅-alkyl radical,
and the salts, diastereomers and enantiomers thereof.

5. Compounds of the formula (I) according to claim 1, in which
X is —O— or —NR¹⁵—, where,
R¹⁵ is hydrogen or a C₃-C₆-alkyl radical, C₃-C₇-cycloalkyl or a heterocyclyl ring having 3 to 6 ring atoms, in each case optionally substituted one or more times, identically or differently, by hydroxy, —NR¹⁰R¹¹, cyano, halogen, —CF₃, C₁-C₆-alkoxy and/or —OCF₃,
or
if X is —NR¹⁵—,
—NR¹⁵— and R² alternatively together form a 5 or 6 membered ring which, in addition to the nitrogen atom, optionally comprises a further heteroatom and is optionally substituted one or more times, identically or differently, by hydroxy, C₁-C₆-alkyl, C₁-C₆-alkoxy, —C(O)R¹², —SO₂R¹², halogen or the group —NR⁸R⁹,
and the salts, diastereomers and enantiomers thereof.

6. Compounds of the formula (I) according to claim 1, in which
R⁴ and R⁵ are independently of one another hydrogen, a C₁-C₃-alkyl radical, —NR⁸R⁹, —OR⁸ or halogen, where R⁸ and R⁹ are independently of one another hydrogen, a monocyclic heteroaryl ring or a C₁-C₆-alkyl radical which are optionally substituted one or more times, identically or differently, by hydroxy, —NR¹⁰R¹¹ or —NR⁷—C(O)—R¹²,
and the salts, diastereomers and enantiomers thereof.

7. Compounds of the formula (I) according to claim 1, in which
R⁶ is a C₂-C₅-alkyl, C₄-C₆-alkenyl, C₄-C₆-alkynyl or C₂-C₅-alkoxy radical, a C₄-C₆-cycloalkyl or phenyl ring, a heterocyclyl ring having 3 to 5 ring atoms or a monocyclic heteroaryl ring, in each case optionally themselves substituted one or more times, identically or differently, by hydroxy, —NR⁸R⁹, cyano, halogen, —CF₃, C₁-C₆-alkoxy and/or —OCF₃,
and the salts, diastereomers and enantiomers thereof.

8. Compounds of the formula (I) according to claim 1, in which
Y is —O— or —NR¹⁵—, where
R¹⁵ is hydrogen or a C₁-C₃-alkyl radical, C₃-C₇-cycloalkyl or a heterocyclyl ring having 3 to 6 ring atoms, in each case optionally substituted one or more times, identically or differently, by hydroxy, —NR¹⁰R¹¹, cyano, halogen, —CF₃, C₁-C₆-alkoxy and/or —OCF₃,
and the salts, diastereomers and enantiomers thereof.

9. Compounds of the formula (I) according to claim 1, in which
R⁸ and R⁹ are independently of one another hydrogen, a monocyclic heteroaryl ring or a C₁-C₆-alkyl radical, which are optionally substituted one or more times, identically or differently, by hydroxy, —NR¹⁰R¹¹ or —NR⁷—C(O)—R¹²,
and the salts, diastereomers and enantiomers thereof.

10. Compounds of the formula (I) according to claim 1, in which
R¹⁰ and R¹¹ are independently of one another hydrogen or a C₁-C₄-alkyl radical which is optionally substituted one or more times, identically or differently, by hydroxy,
and the salts, diastereomers and enantiomers thereof.

11. Compounds of the formula (I) according to claim 1, in which
R¹² is a C₁-C₅-alkyl, C₂-C₅-alkenyl, a C₃-C₆-cycloalkyl or phenyl ring, a heterocyclyl ring having 3 to 6 ring atoms or a monocyclic heteroaryl ring, in each case optionally themselves substituted one or more times, identically or differently, by hydroxy, halogen, nitro, —NR⁸R⁹, C₁-C₆-alkyl and/or C₁-C₆-alkoxy,
and the salts, diastereomers and enantiomers thereof.

12. Compounds of the formula (I) according to claim 1, in which
R¹³ and R¹⁴ are independently of one another a C₁-C₅-alkyl, C₂-C₅-alkenyl and/or C₂-C₅-alkynyl radical, a C₃-C₆-cycloalkyl and/or phenyl ring, a heterocyclyl ring having 3 to 6 ring atoms and/or a monocyclic heteroaryl ring,
and the salts, diastereomers and enantiomers thereof.

13. Compounds of the formula (I) according to claim 1, in which
m is 0 or 1,
and the salts, diastereomers and enantiomers thereof.

14. Compounds of the formula (I) according to claim 1, in which
R¹⁶ is a C₁-C₆-alkyl radical, a C₃-C₇-cycloalkyl or phenyl ring, a heterocyclyl ring having 3 to 8 ring atoms or a monocyclic heteroaryl ring,
and the salts, diastereomers and enantiomers thereof.

15. Compounds according to claim 1 of the general formula (I) in which
R¹ is halogen or —CF₃,
R² is a C₁-C₁₀-alkyl radical which is optionally substituted one or more times, identically or differently, by hydroxy, —NR⁸R⁹, —NR⁷—C(O)—R¹² and/or a monocyclic heteroaryl, optionally itself substituted one or more times by a C₁-C₆-alkyl,
m is 0,
R⁴ and R⁵ are independently of one another hydrogen, —NR⁸R⁹, —OR⁸, halogen, —(CO)—NR⁸R⁹ and/or a C₁-C₆-alkyl radical,
X and Y are independently of one another —O—, —S—, —S(O)— or —NR¹⁵—, where R¹⁵ is hydrogen or a C₁-C₆-alkyl radical,
Q is a monocyclic or bicyclic heteroaryl ring,
R⁷ is hydrogen,
R⁸ and R⁹ are independently of one another hydrogen, a C₁-C₆-alkyl radical and/or a monocyclic heteroaryl ring, which are optionally substituted by hydroxy, —NR¹⁰R¹¹, —NR⁷—C(O)—R¹²,
R¹⁰ and R¹¹ are independently of one another hydrogen or a C₁-C₆-alkyl radical,
R¹² is a C₁-C₆-alkyl radical,
and the salts, diastereomers and enantiomers thereof.

16. Compounds according to claim 1 of the general formula (I) in which
R¹ is halogen,
R² is a C₁-C₁₀-alkyl radical which is optionally substituted one or more times, identically or differently, by hydroxy, —NR⁸R⁹, —NR⁷—C(O)—R¹²
m is 0,
R⁴ and R⁵ are independently of one another hydrogen, —NR⁸R⁹, —OR⁸, halogen, —(CO)—NR⁸R⁹ and/or a C₁-C₆-alkyl radical,
X is —O— or —NR¹⁵—, where R¹⁵ is hydrogen or a C₁-C₆-alkyl radical,
Y is —NR¹⁵—, where R¹⁵ is hydrogen or a C₁-C₆-alkyl radical,
Q is a monocyclic or bicyclic heteroaryl ring,
R⁷ is hydrogen,
R⁸ and R⁹ are independently of one another hydrogen, a C₁-C₆-alkyl radical and/or a monocyclic heteroaryl ring, which are optionally substituted by hydroxy, —NR¹⁰R¹¹, —NR⁷—C(O)—R¹²,
R¹⁰ and R¹¹ are independently of one another hydrogen or a C₁-C₆-alkyl radical,
R¹² is a C₁-C₆-alkyl radical,
and the salts, diastereomers and enantiomers thereof.

17. Compounds of the general formula I according to claim 1 for use as medicaments.

18. Use of compounds of the general formula I according to claim 1 for the manufacture of a medicament for the treatment of cancer.

19. Process for preparing a compound according to claim 1, characterized by the steps:
a) 2,4-dichloropyrimidines of the formula (X) are reacted with nucleophiles of the formula (IX) to give compounds of the formula (II) 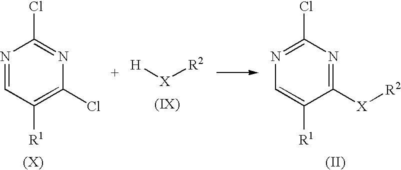
b) 2-chloropyrimidines of the formula (II) are reacted with phenylenediamines of the formula (IIIa) to give compounds of the formula (IV) 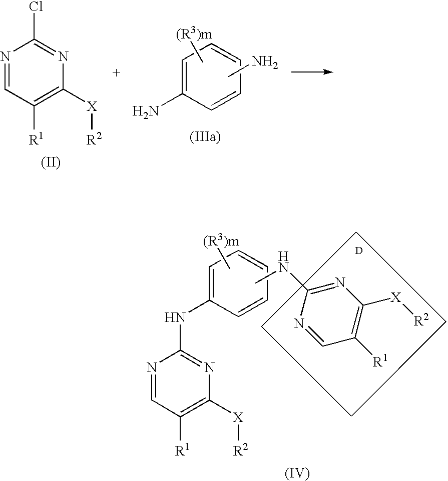
where the substituents R¹, R², R³, X and m have the meanings indicated in general formula (I), and in building block D of the formula (IV) X—R² has the meaning of R⁴ and R¹ has the meaning of R⁵.

20. Process for preparing a compound according to claim 1, characterized by the steps:
a) 2,4-dichloropyrimidines of the formula (X) are reacted with nucleophiles of the formula (IX) to give compounds of the formula (II) 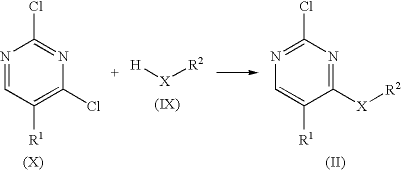
b₁) aniline derivatives of the formula III are reacted with electrophiles of the formula VII 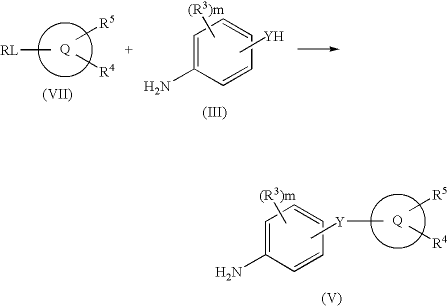
b₂) or alternatively to b₁) for compounds with Y═NH, nitroanilines of the formula (IIIc) are reacted with electrophiles of the formula (VII) and then the nitro group is reduced; 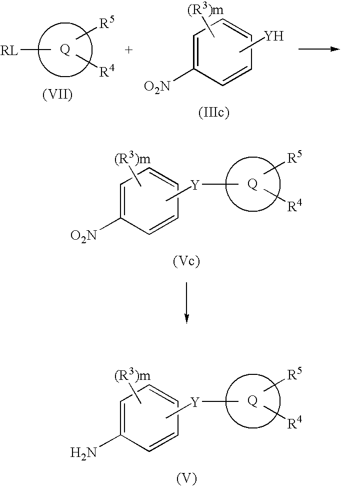
c) 2-chloropyrimidines of the formula (II) from process step a) are reacted with substituted anilines of the formula (V) from process step b₁ or b₂ to give compounds if the formula (I). 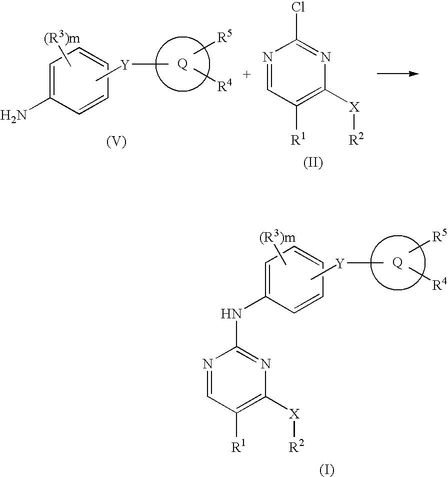
where the substituents Q, R¹, R², R³, R⁴, R⁵, X, Y and m have the meanings indicated in the general formula (I).

21. Process for preparing a compound according to claim 1, characterized by the steps:
a) 2,4-dichloropyrimidines of the formula (X) are reacted with nucleophiles of the formula (IX) to give compounds of the formula (II) 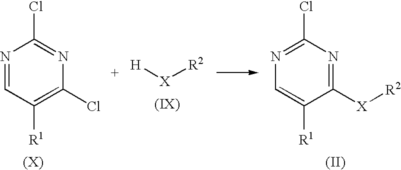
b₁) 2-chloropyrimidines of the formula (II) are reacted with nucleophiles of the formula (IIId) to give compounds of the formula (VI). 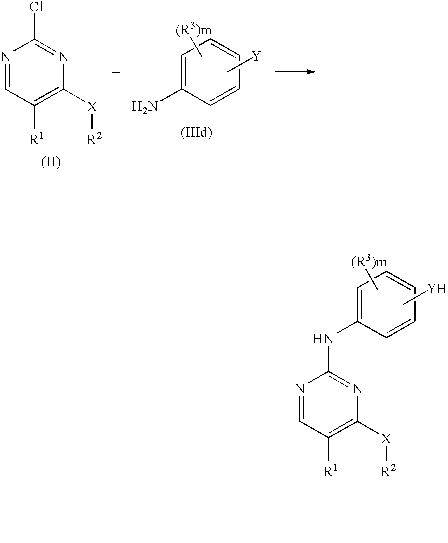
b₂) or alternatively to b₁) for compounds with Y═NH, 2-chloropyrimidines of the formula (II) are reacted with nitroanilines of the formula (IIIc) to give compounds (VIb), and then the nitro group is reduced 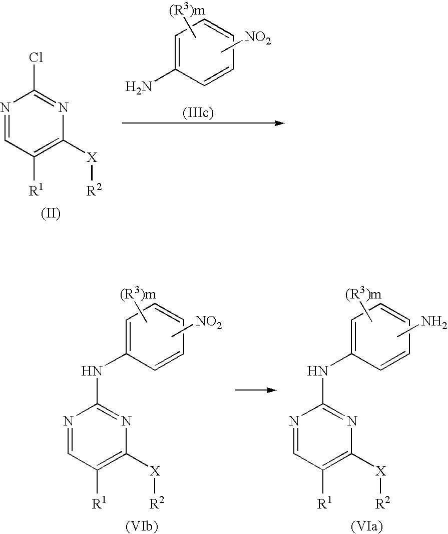
c) substituted anilinopyrimidines of the formula (VI) or (VIa) from process steps b₁ or b₂ are reacted with electrophiles of the formula (VII) to give compounds of the formula (I) 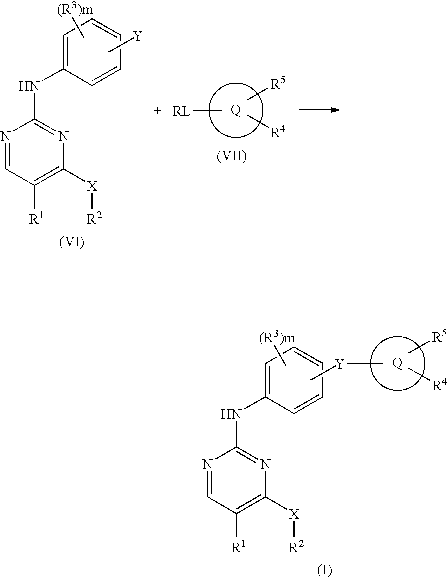
where the substituents Q, R¹, R², R³, R⁴, R⁵, X, Y and m have the meanings indicated in the general formula (I), and RL is a leaving group.

22. Process for preparing a compound according to claim 1, characterized by the steps:
a₁) aniline derivatives of the formula III are reacted with electrophiles of the formula VII 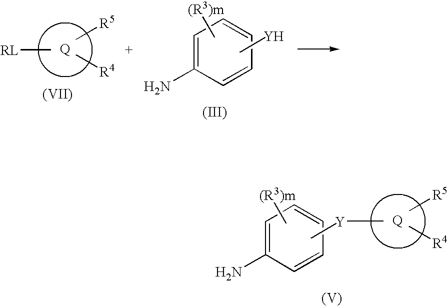
a₂) or alternatively nitroanilines of the formula (IIIc) are reacted with electrophiles of the formula (VII), and then the nitro group is reduced, 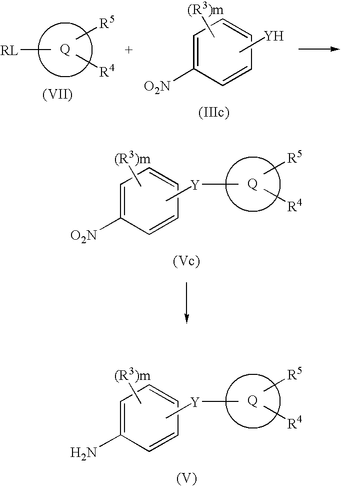
b) substituted 2-chloropyrimidines of the formula (XI) are reacted with nucleophiles of the formula (V) from process steps a₁ or a₂ to give compounds of the formula (VIII). 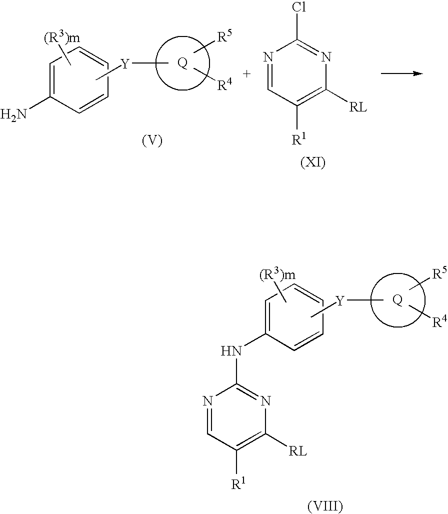
c) substituted anilinopyrimidines of the formula (VIII) are reacted with nucleophiles of the formula (IX) to give compounds of the formula (I). 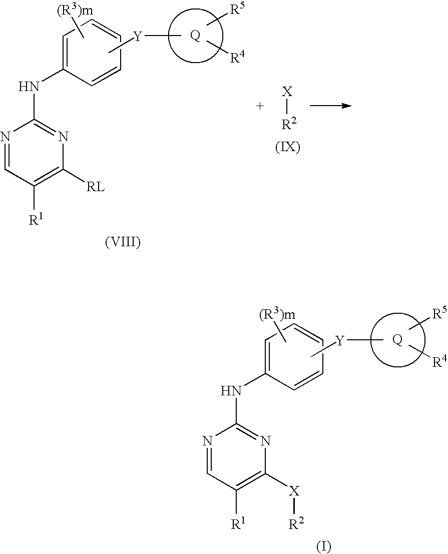
where the substituents Q, R¹, R², R³, R⁴, R⁵, X, Y and m have the meanings indicated in the general formula (I), and RL is a leaving group.

23. Pharmaceutical formulation comprising one or more compounds according to claim 1.

24. A method of treating cancer comprising administering a compound of claim 1.
